SEQUENCE LISTING (66 sequences)

SEQ ID NO: 1 - DNA, 40 bp, Artificial sequence
  Single strand DNA oligonucleotide
tggtgggtcctccggattacaggcgtgcaaaatggaaggg

SEQ ID NO: 2 - DNA, 43 bp, Artificial sequence
  Single strand DNA oligonucleotide
tggtgggtcctccggagacgggactattaaggaggctctgtcg

SEQ ID NO: 3 - DNA, 44 bp, Artificial sequence
  Single strand DNA oligonucleotide
aaacgggccctctagactagtagtagctgcctaagtgtgaaggc

SEQ ID NO: 4 - DNA, 38 bp, Artificial sequence
  Single strand DNA oligonucleotide
tggtgggtcctccggagagagtgcaatcacgctgtggc

SEQ ID NO: 5 - DNA, 49 bp, Artificial sequence
  Single strand DNA oligonucleotide
aaacgggccctctagatcaggatttctgagagtttgaagaaagcagtac

SEQ ID NO: 6 - DNA, 39 bp, Artificial sequence
  Single strand DNA oligonucleotide
tggtgggtcctccggaattcagactgtcccggacccagc

SEQ ID NO: 7 - DNA, 45 bp, Artificial sequence
  Single strand DNA oligonucleotide
aaacgggccctctagattagtagtaagtgcccagatgagaaggca

SEQ ID NO: 8 - DNA, 41 bp, Artificial sequence
  Single strand DNA oligonucleotide
tggtgggtcctccggagctgcaacctgtgagattagcaaca

SEQ ID NO: 9 - DNA, 44 bp, Artificial sequence
  Single strand DNA oligonucleotide
aaacgggccctctagatcagttccgactctggagaacctcttcc

SEQ ID NO: 10 - DNA, 42 bp, Artificial sequence
  Single strand DNA oligonucleotide
tggtgggtcctccggaacgtgtgttgaacaagacaagctggg

SEQ ID NO: 11 - DNA, 51 bp, Artificial sequence
  Single strand DNA oligonucleotide
aaacgggccctctagattagcaatcatggtgatttagctcatggtaattgg

SEQ ID NO: 12 - DNA, 39 bp, Artificial sequence
  Single strand DNA oligonucleotide
tggtgggtcctccggagacctgtggaactgggatgaggc

SEQ ID NO: 13 - DNA, 41 bp, Artificial sequence
  Single strand DNA oligonucleotide
aaacgggccctctagattattgtgtctctgctccccgtccg

SEQ ID NO: 14 - DNA, 40 bp, Artificial sequence
  Single strand DNA oligonucleotide
tggtgggtcctccggagacagtgctatcaccctgtggcag

SEQ ID NO: 15 - DNA, 47 bp, Artificial sequence
  Single strand DNA oligonucleotide
aaacgggccctctagattatgtcttctgtaggtctggggaaaatggg

SEQ ID NO: 16 - DNA, 46 bp, Artificial sequence
  Single strand DNA oligonucleotide
tggtgggtcctccggaactaaaagagaagcagaggagctgatagaa

SEQ ID NO: 17 - DNA, 46 bp, Artificial sequence
  Single strand DNA oligonucleotide
aaacgggccctctagatcaattatccttttccgtttgcagagaagc

SEQ ID NO: 18 - DNA, 38 bp, Artificial sequence
  Single strand DNA oligonucleotide
tggtgggtcctccggagacccatctgtgacgctgtggc

SEQ ID NO: 19 - DNA, 37 bp, Artificial sequence
  Single strand DNA oligonucleotide
aaacgggccctctagatcatggcttctggggccctgg

SEQ ID NO: 20 - DNA, 40 bp, Artificial sequence
  Single strand DNA oligonucleotide
tggtgggtcctccggagagcggaggatgaaagccggatac

SEQ ID NO: 21 - DNA, 42 bp, Artificial sequence
  Single strand DNA oligonucleotide
aaacgggccctctagactagtaagagtagccacccttggggc

SEQ ID NO: 22 - DNA, 43 bp, Artificial sequence
  Single strand DNA oligonucleotide
tggtgggtcctccggacaggagggagaattggctatttctcct

SEQ ID NO: 23 - DNA, 42 bp, Artificial sequence
  Single strand DNA oligonucleotide
aaacgggccctctagatcacggagagatttctggcctcttgt

SEQ ID NO: 24 - DNA, 43 bp, Artificial sequence
  Single strand DNA oligonucleotide
tggtgggtcctccggagacgggttttatgatcagcaagtccct

SEQ ID NO: 25 - DNA, 44 bp, Artificial sequence
  Single strand DNA oligonucleotide
aaacgggccctctagattagtaagcaaagccttcggcatagggg

SEQ ID NO: 26 - DNA, 46 bp, Artificial sequence
  Single strand DNA oligonucleotide
tggtgggtcctccggatctgagactcctgctcagtgtagcattaag

SEQ ID NO: 27 - DNA, 52 bp, Artificial sequence
  Single strand DNA oligonucleotide
aaacgggccctctagatcagcattcatcttcttggtatatttgttcatccag

SEQ ID NO: 28 - DNA, 43 bp, Artificial sequence
  Single strand DNA oligonucleotide
tggtgggtcctccggaagctactttgtggattctgctgggagc

SEQ ID NO: 29 - DNA, 40 bp, Artificial sequence
  Single strand DNA oligonucleotide
aaacgggccctctagatcactcgtcggcatctggcttgac

SEQ ID NO: 30 - DNA, 37 bp, Artificial sequence
  Single strand DNA oligonucleotide
tggtgggtcctccggagggtcggctcaatttcagggc

SEQ ID NO: 31 - DNA, 35 bp, Artificial sequence
  Single strand DNA oligonucleotide
aaacgggccctctagatcagtcctccgtgtcgggc

SEQ ID NO: 32 - DNA, 41 bp, Artificial sequence
  Single strand DNA oligonucleotide
tggtgggtcctccggagacgaactgttccccctcatcttcc

SEQ ID NO: 33 - DNA, 40 bp, Artificial sequence
  Single strand DNA oligonucleotide
aaacgggccctctagatcacccccttaggagctgatctga

SEQ ID NO: 34 - DNA, 51 bp, Artificial sequence
  Single strand DNA oligonucleotide
agcacagtggcggccgcatggactccaaagaatcattaactcctggtagag

SEQ ID NO: 35 - DNA, 50 bp, Artificial sequence
  Single strand DNA oligonucleotide
ccaccgccaccatcgatcttttgatgaaacagaagttttttgatatttcc

SEQ ID NO: 36 - DNA, 45 bp, Artificial sequence
  Single strand DNA oligonucleotide
agcacagtggcggccgcatggagaccaaaggctaccacagtctcc

SEQ ID NO: 37 - DNA, 39 bp, Artificial sequence
  Single strand DNA oligonucleotide
ccaccgccaccatcgatcttccggtggaagtagagcggc

SEQ ID NO: 38 - DNA, 43 bp, Artificial sequence
  Single strand DNA oligonucleotide
agcacagtggcggccgcatgaccatgaccctccacaccaaagc

SEQ ID NO: 39 - DNA, 45 bp, Artificial sequence
  Single strand DNA oligonucleotide
ccaccgccaccatcgatgaccgtggcagggaaaccctctgcctcc

SEQ ID NO: 40 - DNA, 59 bp, Artificial sequence
  Single strand DNA oligonucleotide
agcacagtggcggccgcatggatataaaaaactcaccatctagccttaattctccttcc

SEQ ID NO: 41 - DNA, 50 bp, Artificial sequence
  Single strand DNA oligonucleotide
ccaccgccaccattcgatctgagactgtgggttctgggagccctctttgc

SEQ ID NO: 42 - DNA, 62 bp, Artificial sequence
  Single strand DNA oligonucleotide
agcacagtggcggccgcatggactccaaagaatcattaactcctggtagagaagaaaacccc

SEQ ID NO: 43 - DNA, 62 bp, Artificial sequence
  Single strand DNA oligonucleotide
  misc_feature (59)..(59): n is a, c, g, or t
ccaccgccaccatcgatcttgaatagccattagaaaaaactgttcgaccagggsamasntdw

SEQ ID NO: 44 - DNA, 47 bp, Artificial sequence
  Single strand DNA oligonucleotide
ccaccgccaccatcgatcttccaggttcattccagcctgaagacatt

SEQ ID NO: 45 - DNA, 50 bp, Artificial sequence
  Single strand DNA oligonucleotide
agcacagtggcggccgcatgagccccagcatgagaccagatgtaagctct

SEQ ID NO: 46 - DNA, 65 bp, Artificial sequence
  Single strand DNA oligonucleotide
ccaccgccaccatcgatcttttgatgaaacagaagttttttgatatttccatttgaatattttgg

SEQ ID NO: 47 - DNA, 75 bp, Artificial sequence
  Single strand DNA oligonucleotide
agcacagtggcggccgcatggctcgaaaaacaaagaaaaaaataaaaggaattcagcaggcsamasdbdhrbdrv

SEQ ID NO: 48 - DNA, 44 bp, Artificial sequence
  Single strand DNA oligonucleotide
tggtgggtcctccggagacgggactattaaggaggctctgtcgg

SEQ ID NO: 49 - DNA, 62 bp, Artificial sequence
  Single strand DNA oligonucleotide
  misc_feature (57)..(57): n is a, c, g, or t
aaacgggccctctagattatttccctgaggtaactgaggtgtgacaacagcsamasntatsw

SEQ ID NO: 50 - DNA, 60 bp, Artificial sequence
  Single strand DNA oligonucleotide
  misc_feature (55)..(55): n is a, c, g, or t
aaacgggccctctagattaggagagcagctccaggaggaattgccacagsamasntatsw

SEQ ID NO: 51 - DNA, 40 bp, Artificial sequence
  Single strand DNA oligonucleotide
aaacgggccctctagattactgctggtgggcatggtagga

SEQ ID NO: 52 - DNA, 22 bp, Artificial sequence
  Single strand DNA oligonucleotide
ggacactgcaaaaacctagagc

SEQ ID NO: 53 - DNA, 23 bp, Artificial sequence
  Single strand DNA oligonucleotide
ccagaacctgattgactgtagca

SEQ ID NO: 54 - DNA, 21 bp, Artificial sequence
  Single strand DNA oligonucleotide
cataaccgctgtgcggaaaat

SEQ ID NO: 55 - DNA, 22 bp, Artificial sequence
  Single strand DNA oligonucleotide
tcatctagggacttaccacctg

SEQ ID NO: 56 - DNA, 20 bp, Artificial sequence
  Single strand DNA oligonucleotide
gcccttttccgttagcgttc

SEQ ID NO: 57 - DNA, 21 bp, Artificial sequence
  Single strand DNA oligonucleotide
acttgcgagagttattgggca

SEQ ID NO: 58 - DNA, 21 bp, Artificial sequence
  Single strand DNA oligonucleotide
gcagtttgttgagccaagtcg

SEQ ID NO: 59 - DNA, 19 bp, Artificial sequence
  Single strand DNA oligonucleotide
ccagccgaatggagtcctt

SEQ ID NO: 60 - DNA, 21 bp, Artificial sequence
  Single strand DNA oligonucleotide
ccctggagtaccgagtcttcc

SEQ ID NO: 61 - DNA, 23 bp, Artificial sequence
  Single strand DNA oligonucleotide
catttttgcattagaccagcgtg

SEQ ID NO: 62 - DNA, 21 bp, Artificial sequence
  Single strand DNA oligonucleotide
cgagcaaggtccttccatagt

SEQ ID NO: 63 - DNA, 20 bp, Artificial sequence
  Single strand DNA oligonucleotide
ggtgtcgggattccgatgac

SEQ ID NO: 64 - DNA, 22 bp, Artificial sequence
  Single strand DNA oligonucleotide
ccaaacgtaccaagaaagtcgg

SEQ ID NO: 65 - DNA, 19 bp, Artificial sequence
  Single strand DNA oligonucleotide
gcgtgctggctgatttcaa

SEQ ID NO: 66 - DNA, 33 bp, Artificial sequence
  Single strand DNA oligonucleotide
aaacgggccctctagatcagtggggcgggtggc

(19) United States
(12) Patent Application Publication
(10) Pub. No.: US 2021/0275545 A1
(43) Pub. Date: Sep. 9, 2021

(54) METHOD OF TREATING CANCER PREFERABLY CHARACTERIZED BY EXPRESSION OF A FUSION PROTEIN COMPRISING A MEMBER OF THE E-TWENTY-SIX FAMILY BY ADMINISTERING AN AGENT THAT INHIBITS THE SYNTHESIS AND/OR ACTIVITY OF CORTISOL

(71) Applicant: Yeda Research and Development Co. Ltd., Rehovot (IL)

(72) Inventors: Yosef YARDEN, Rehovot (IL); Swati SRIVASTAVA, Rehovot (IL)

(73) Assignee: Yeda Research and Development Co. Ltd., Rehovot (IL)

(21) Appl. No.: 17/325,355

(22) Filed: May 20, 2021

Related U.S. Application Data
(63) Continuation of application No. PCT/IL2019/051273, filed on Nov. 21, 2019.

(30) Foreign Application Priority Data
Nov. 21, 2018 (IL) 263184

(51) Int. Cl. A61K 31/567; A61K 31/496; A61K 31/4188; A61K 31/03; A61K 31/451; A61K 31/4174; A61K 31/444; A61K 31/437; A61P 35/00; C12Q 1/6886

(52) U.S. Cl. CPC A61K 31/567; A61K 31/496; A61K 31/4188; A61K 31/03; A61K 31/451; C12Q 2600/158; A61K 31/444; A61K 31/437; A61P 35/00; C12Q 1/6886; A61K 31/4174

(57) ABSTRACT

A method of treating a cancer selected from the group consisting of a myeloid malignancy, a lymphoid malignancy and Ewing's sarcoma is disclosed. The method comprises administering to the subject a therapeutically effective amount of an agent that inhibits the synthesis and/or activity of cortisol.

### RELATED APPLICATIONS

This application is a Continuation of PCT Patent Application No. PCT/IL2019/051273 having International filing date of Nov. 21, 2019, which claims the benefit of priority of Israel Patent Application No. 263184 filed on Nov. 21, 2018. The contents of the above applications are all incorporated by reference as if fully set forth herein in their entirety.

### SEQUENCE LISTING STATEMENT

The ASCII file, entitled 87268SequenceListing.txt, created on May 20, 2021, comprising 14,510 bytes, submitted concurrently with the filing of this application is incorporated herein by reference.

### FIELD AND BACKGROUND OF THE INVENTION

The present invention, in some embodiments thereof, relates to a method of treating cancer and, more particularly, but not exclusively, to cancers that are associated with expression of an oncogenic fusion protein which comprises a member of the E26 transformation-specific (ETS) family.

Endogenous glucocorticoids (GCs) modulate many physiological and cellular processes, including cell proliferation, metabolism, growth arrest and apoptosis. Synthetic GCs like dexamethasone (DEX) have been widely used in the treatment of hematologic malignancies, as a cytotoxic agent, and in the treatment of solid tumors, to prevent complications associated with cancer therapy. The cellular actions of GCs are mediated by the glucocorticoid receptor (GR). Like other nuclear receptors, GR resides in the cytoplasm, stabilized by chaperone proteins. Once bound by GCs, GR homodimers translocate into the nucleus to regulate multiple genes, either positively or negatively. Positive regulation, a mechanism known as GR-dependent transactivation (TA), entails receptor binding to palindromic DNA sequences, called GC response elements (GREs). A similarly important negative regulation is called GR-dependent transrepression (TR). This is mediated by physical interactions between GR and DNA-bound transcription factors (TFs), such as nuclear factor kappa B (NF-κB), Stat5 and the activator protein 1 (AP-1).

Apart from the classic TR mechanism, GR can sequester specific TFs, thereby prevent their binding to the respective response elements. In addition, GR's function is regulated by p53, which helps recruit the transcriptional activation machinery to the promoter regions of GR target genes. Formation of GR/p53 complexes also drives inhibition of p53-dependent functions, including cell cycle arrest and apoptosis, due to cytoplasmic sequestration of both TFs. Interestingly, the ability of GR to interfere with NF-κB and AP-1 can lead to mutual inhibition. Despite mutual antagonism, AP-1 has emerged as a key partner in GR-regulated transcription, by means of enhancing GR binding to specific sites in the genome. Likewise, although recruitment of GR to DNA-bound Stat3 is associated with transcriptional antagonism, the reciprocal recruitment of Stat3 to DNA-bound GR results in transcriptional synergism.

Background art includes US Patent Application No. 20090324587, U.S. Pat. No. 8,658,128; Rastogi et al., Genes Cancer. 2014 July; 5(7-8): 273-284, Sahu et al., Cancer Res; 73(5) Mar. 1, 2013 and Taplin et al., (2008) BJU international. 101. 1084-9.

### SUMMARY OF THE INVENTION

According to an aspect of the present invention there is provided a method of treating a cancer selected from the group consisting of a myeloid malignancy, a lymphoid malignancy and Ewing's sarcoma in a subject in need thereof comprising administering to the subject a therapeutically effective amount of an agent that inhibits the synthesis and/or activity of cortisol, thereby treating the cancer.

According to an aspect of the present invention there is provided an agent that inhibits the synthesis and/or activity of cortisol for use in treating a cancer selected from the group consisting of a myeloid malignancy, a lymphoid malignancy and Ewing's sarcoma.

According to an aspect of the present invention there is provided a method of treating a subject having a cancer characterized by expression of a fusion protein which comprises a member of the E-twenty-six (ETS) family, the method comprising:

(a) analyzing in a sample of the subject for the presence of a genomic ETS rearrangement; and

(b) administering to the subject a therapeutically effective amount of an agent that inhibits the synthesis and/or activity of cortisol upon identification of the genomic ETS rearrangement, thereby treating the cancer.

According to an aspect of the present invention there is provided a method of treating a subject having a cancer characterized by expression of a fusion protein which comprises a member of the E-twenty-six (ETS) family, the method comprising:

(a) analyzing in a sample of the subject for the presence of a genomic ETS rearrangement; and

(b) administering to the subject a therapeutically effective amount of an agent that inhibits the synthesis and/or activity of cortisol upon identification of the genomic ETS rearrangement, or administering to the subject a therapeutically effective amount of an anti-cancer agent other than an agent that inhibits the synthesis and/or activity of cortisol upon identification of an absence of the genomic ETS rearrangement, thereby treating the cancer.

According to an aspect of the present invention there is provided a method of selecting a treatment for a subject having a cancer characterized by expression of a fusion protein which comprises a member of the E26 transformation-specific (ETS) family, the method comprising analyzing in a sample of the subject for the presence of a genomic ETS rearrangement, wherein the presence of the genomic ETS rearrangement is indicative that the subject should be treated with an agent that inhibits the synthesis and/or activity of cortisol.

According to an aspect of the present invention there is provided an agent that inhibits the synthesis and/or activity of cortisol for use in treating a cancer characterized by expression of a fusion protein which comprises a member of the E-twenty-six (ETS) family.

According to an aspect of the present invention there is provided a method of predicting survival of a subject having a Ewing's sarcoma comprising analyzing in a sample of the subject for the presence of cells having a signature comprising each of PDIA6, COL6A3, TMED10, SEC61G, PPA1, IGFBP7, and RPL37A, wherein the signature is indicative of short survival.

According to embodiments of the present invention, the sample comprises a fluid sample.

According to embodiments of the present invention, the fluid sample is selected from the group consisting of whole blood, plasma, serum and urine.

According to embodiments of the present invention, the sample comprises a tissue sample.

According to embodiments of the present invention, the analyzing for the presence of the genomic ETS rearrangement is effected at the DNA level.

According to embodiments of the present invention, the analyzing for the presence of the genomic ETS rearrangement is effected at the RNA level.

According to embodiments of the present invention, the analyzing for the presence of the genomic ETS rearrangement is effected at the protein level.

According to embodiments of the present invention, the cancer is selected from the group consisting of myeloid malignancy, a lymphoid malignancy, prostate cancer and Ewing's sarcoma.

According to embodiments of the present invention, the member of the ETS family is ERG or FL1.

According to embodiments of the present invention, the subject expresses a genomic ETS rearrangement.

According to embodiments of the present invention, the agent that inhibits the activity of cortisol is a glucocorticoid receptor antagonist.

According to embodiments of the present invention, the glucocorticoid receptor antagonist is a selective inhibitor of the glucocorticoid receptor.

According to embodiments of the present invention, the selective inhibitor of the glucocorticoid receptor is C113176 or C108297.

According to embodiments of the present invention, the glucocorticoid receptor antagonist comprises a steroidal back-bone.

According to embodiments of the present invention, the glucocorticoid receptor antagonist is mifepristone.

According to embodiments of the present invention, the glucocorticoid receptor antagonist comprises a non-steroidal back-bone.

According to embodiments of the present invention, the agent that inhibits synthesis of cortisol is selected from the group consisting of metyrapone ketoconazole, levoketoconazole, LCI699, mitotane, aminoglutethimide and etomidate.

According to embodiments of the present invention, the agent is metyrapone.

According to embodiments of the present invention, the agent that inhibits synthesis of cortisol is a polynucleotide agent or a proteinaceous agent that targets a component of the cortisol synthesis pathway.

According to embodiments of the present invention, the component is 11-beta-hydoxylase.

According to embodiments of the present invention, the sample comprises a tumor sample.

According to embodiments of the present invention, the sample comprises a body fluid.

According to embodiments of the present invention, the body fluid comprises whole blood, serum, plasma and urine.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E: Protein-fragment complementation assays (PCA) detects physical interactions between GR and specific members of the ETS family. (A) The scheme presents the full length Gaussia luciferase protein, along with either the amino-terminal segment (Gluc1), which was fused to an ETS family transcription factor, or the carboxyl terminal segment (Gluc2), which was fused to GR. Amino acid numbers are indicated. cDNAs encoding most members of the ETS family (16 members altogether) were cloned downstream to Gluc1. Note that in isolation both Gluc1 and Gluc2 fragments are structurally unfolded and inactive. However as soon as a suitable ligand ETS-TF comes into close proximity with GR, the inactive fragments might regain their conformation and activity, which can be quantified as a luminescence signal. (B) HEK-293T cells (6×10³) were seeded in 96-well plates. On the next day, cells were transfected with combinations of the Gluc1 plasmid, encoding a fused ETS family member, and the Gluc2 plasmid encoding a fused full-length GR. After 24 hours, cells were starved overnight for serum factors and thereafter they were treated with vehicle or with Dexamethasone (DEX; 1 μM) for 60 minutes. Cells were then lysed and luminescence was determined in biological triplicates. The bar plot shows the normalized, DEX-induced fold changes in luciferase activity for each ETS family member (means±S.E.; **, p≤0.005; ***, p≤0.001. (C) HEK-293T cells were co-transfected in sextuplicates with GR-Gluc2 and either Gluc1-FLI1, Gluc1-PU.1, Gluc-1-ERG or Gluc-1-ETV4. Cells were later treated with either vehicle, DEX (1 μM), or a combination of DEX and RU486 (1 μM), and PCA was performed. Bar plots show the fold change in luminescence in response to DEX alone or DEX+RU486 (means±S.E.). Luminescence signals corresponding to vehicle-treated cells were subjected to normalization. **, p≤0.005; ***, p≤0.001; ns, not significant. (D and E) HEK-293T cells were seeded in 100-mm dishes. Once they reached sub-maximal density (70%), cells were starved overnight for serum factors. Thereafter, they were treated for 60 minutes with vehicle, DEX, RU486 or the combination. Cell extracts were then processed for co-immunoprecipitation assays, in which the endogenous FLI1 or ERG protein was immunoprecipitated using a specific antibody, and immunoblotting was performed using an antibody against GR. EB, empty beads; C, solvent control. Images shown are representative of three biological replicates.

FIGS. 2A-C: Following treatment with DEX, GR translocates to the nucleus, FLI1 follows and forms a transient physical complex with GR. (A) HEK-293T cells (6×10³) were seeded in 96-well plates. On the next day, the cells were transfected with combinations of the Gluc1 plasmid, encoding fused FLI1 or ERG, and the Gluc2 plasmid encoding GR. After 24 hours, cells were starved overnight for serum factors and thereafter they were treated with vehicle or with DEX (1 μM) for the indicated time periods. Cells were then lysed and luminescence was determined in each sample. Luminescence of vehicle-treated cells was used for normalization. The bar plot shows the DEX-induced normalized fold changes in luciferase activity for each set of interactions between an ETS family member and GR. *, p≤0.05; **, p≤0.01 (n=3). (B) HEK-293T cells were seeded on coverslips in 6-well plates. After 24 hours, cells were starved overnight for serum factors, followed by treatment with DEX (1 μM) for the indicated time periods. The cells were washed in saline containing tween 20 (0.01%; w/v), fixed overnight at 4° C. in formaldehyde (4%; in saline) and permeabilized for 7 minutes. Prior to probing with the primary antibody, non-specific sites were blocked using fetal bovine serum. The incubation with the primary antibody (overnight at 4° C.) was followed by an FITC-conjugated secondary antibody and DAPI (45 minutes in dark). After three washes, cells were mounted on microscope slides and images were captured using a Zeiss confocal microscope (40× magnification). Images were processed using the Zeiss ZEN2011 software. Quantification of nuclear localization (average±S.E.) surveyed 180 cells from each condition. Bars, 20 μm. (C) HEK-293T cells grown in 6-well plates were starved overnight for serum factors, and later treated with DEX (1 μM) for the indicated time periods. The cells were later harvested, sedimented using a centrifuge and resuspended in buffer prior to fractionation into cytoplasmic and nuclear fractions. Each fraction was resolved by gel electrophoresis followed by immunoblotting that used the indicated antibodies.

FIGS. 3A-D Mapping the mutually interacting domains of FLI1 and GR. Schematic diagrams showing the domain structures of FLI1 (A) and GR (B). Different domains of FLI1 were inserted C-terminally to GLuc1. Likewise, individual domains of GR were inserted N-terminally to Gluc2. HEK-293T cells (6×10³) were seeded in each well of 96-well plates. On the next day, cells were co-transfected with the Gluc1 plasmid (A) encoding different domains of FLI1 and the Gluc2 plasmid encoding full length GR. Alternatively, we used the Gluc2 plasmid (B) encoding different domains of GR, and the Gluc1 plasmid encoding the full length FLI1. After 24 hours, cells were starved overnight for serum factors and later treated with vehicle or with DEX (1 μM) for 60 minutes. The cells were then lysed and luminescence was determined in biological triplicates. The bar plots show the fold change in luciferase activity induced by DEX, relative to vehicle-treated cells. *, p≤0.05; **, p≤0.01. (C and D) HEK-293T cells were transfected with Gluc1 plasmids encoding different domains of FLI1 (C), or Gluc2 plasmids encoding different domains of GR. Cells were lysed after 24 hours and processed for co-immunoprecipitation assays, in which the Gluc protein was immunoprecipitated using a specific antibody. Immunoblotting was performed using antibodies that detected the endogenous form of the respective interacting protein, either GR or FLI1. Blots are representative of three or more biological replicates. For reference, we present immunoblots that used Gluc antibodies and different antibodies to GR. In addition, the lowermost panels present immunoblots of whole cell extracts (no prior immunoprecipitation) blotted for the respective endogenous protein, either GR (C) or FLI1 (D). Similarly, the input of recombinant proteins is shown in the middle panels of C and D. Both molecular weights and relative quantities of individual Gluc fusion proteins are shown by means of immunoblotting with an antibody to Gluc. Individual fusion proteins are identified by numbers and their molecular weights are calculated below each panel.

FIGS. 4A-F: FLI1 enhances transcription from the GRE. HEK-293T cells (1.2×10⁴) were seeded in 48-well plates. On the next day, cells were transfected with a reporter plasmid encoding GRE-luciferase (2.5 μg). In addition, cells were co-transfected with increasing amounts of a GR expression vector (A), a FLI1-encoding vector (B) or with a combination of both a FLI1 vector and a vector encoding GR, either wild type (C) or a mutant receptor (GRdim−, panel D). Luciferase activity was determined, in biological triplicates, 48 hours later and presented in bar plots. Cells that were pre-transfected with the reporter plasmid were used for normalization. *, p≤0.05; ***, p≤0.001. (E) HEK-293T cells were co-transfected with a GRE-luciferase plasmid and the indicated amounts of a FLI1 expression vector. After 24 hours, cells were starved overnight for serum factors and treated for 24 hours with DEX, RU486 (each at 100 nM) or with the combination of drugs. This was followed by cell lysis and determination of luciferase activity in biological triplicates. (F) A schematic diagram presenting GR dimers occupying the GRE, either before or after recruiting FLI1 proteins, which enhance the transcriptional activity of GR.

FIGS. 5A-H: Ligand-induced activation of GR in Ewing's sarcoma cells is associated with increased cellular migration and invasion. (A) CHLA9 cells were seeded in 100-mm dishes. Once they reached sub-maximal density, cells were starved overnight for serum factors. Thereafter, they were treated for 60 minutes with vehicle, DEX (1 μM), RU486 (1 μM), or with their combination. Cells were then extracted and the lysates were processed for co-immunoprecipitation assays using an antibody against GR followed by immunobloting that used an antibody against EWS. The image shown is representative of 3 biological replicates. Signals were quantified and normalized (see numbers below each lane). EB, empty beads. (B) CHLA9 cells were transfected with either GR-specific or control scrambled siRNAs (siC). GR knockdown efficiency was tested after 48 hours using immunoblotting with antibodies to GR or ERK. (C-D) CHLA9 cells were seeded on the upper faces of either Transwell migration chambers or Matrigel-coated invasion chambers, and further incubated for 20 hours in full medium. Control siRNAs or siRNAs specific to either GR or EWS-FLI1 were added to cells 24 hours prior to seeding, and both cell migration and invasion were measured 20 hours later. Alternatively, DEX (1 μM), RU486 (1 μM) or the combination of drugs were added to the medium and migration/invasion were assayed 20 hours later. Cells that reached the lower faces of the chambers were fixed and stained. Shown are representative images of stained cells. The bar plots show quantification, using ImageJ, of the area covered by cells. *, p≤0.05, **, p≤0.005. Bars, 500 μm. (E) CHLA9 cells were transfected with either FLI1-specific or control scrambled siRNAs (siC). FLI1 knockdown efficiency was tested after 48 hours using immunoblotting. Note that the EWS-FLI1 fusion protein (ca. 65 kDa) was more effectively downregulated than the endogenous form of FLI1 (ca. 50 kDa). (F) CHLA9 cells were treated with siRNAs as in E. Twenty-four hours later, cells were seeded on the upper faces of Transwell migration chambers and further incubated for 20 hours in full medium. Thereafter, cells were treated with DEX (1 μM) and their migration was assayed 20 hours later. Cells that reached the lower faces of the chambers were fixed and stained. Shown are representative images of stained cells. The bar plots show quantification, using ImageJ, of the area covered by cells. *, p≤0.05, **, p≤0.005. Bars, 500 μm. (G) Plates were pre-coated with fibronectin. (Cultrex® RGF BME, Trevigen Inc.) and then CHLA9 cells, which were pre-transfected with si-GR or Si—C, were seeded and allowed to attach for 90 minutes. Unattached cells were removed and adherent cells were fixed with paraformaldehyde (4%), stained in crystal violet solution (0.1%) and their optical density (550 nm) was quantified in triplicates. *, p<0.01. (H) CHLA9 cells were transfected with vectors encoding the following proteins: GR, FLI1, GRdim⁻ (A458T) or the indicated combinations. Twenty-four hours later, cells were seeded on porous filters as described in C and their migration and invasion were assayed. Quantification of the signals (means±S.D. of duplicates) and representative microscope fields are presented. *, p≤0.05; **p≤0.005; ***, p≤0.001. ns, not significant. Bars, 500 μm (migration) or 100 μm (invasion). The assay was repeated twice.

FIGS. 6A-E. A GR antagonist and a cortisol-lowering drug impede growth of human xenograft models of Ewing's sarcoma. (A) RD-ES cells (2×10⁶) were implanted subcutaneously in the right flank of SCID mice. Once tumors reached an approximate size of 150 mm³, animals were randomized into three groups (n=10). Each group was daily treated with either vehicle, DEX (1 mg/kg) or RU486 (1 mg/kg) and tumorigenic growth was monitored. The means of tumor volumes (+SEM) are shown. Statistical analysis of tumor volumes determined on day 12 is indicated (RU486 group vs. DEX or Vehicle). (B) Shown are representative images of immunofluorescent staining for KI67 in paraffin-embedded sections of tumors from A, using an antibody against KI67. Scale bars, 100 μm. Also shown is the scatter plot depicting quantification of KI67 staining in randomly selected 4 fields of a representative tumor from each group. Note: p<0.01 for control group vs. RU486. (C and D) A673 cells (2×10⁶) were implanted in female SCID mice. Once tumors reached the size of 150 mm³, animals were randomized into two groups (n=9). Each group was daily treated with either vehicle, or Metyrapone (25 mg/kg) and tumorigenic growth was monitored. Actual tumor volumes (±SEM) are presented. Also shown are tumors harvested from each group of animals; **p<0.001. (E) TC-71 cells (10⁶) engineered to stably express luciferase were injected into the tibia of female SCID mice (5 weeks old; 7 mice per group). One week later, mice were treated intraperitoneally with DEX or RU486 (each at 1 mg/kg). The control group was similarly treated with the solvent (0.1% tween 80). When the primary tumor reached 10% of body weight, the lungs were excised and examined for metastasis using luciferase bioluminescence imaging. Shown are whole lungs from representative animals from each group. Luminescence was quantified and presented in a bar graph. Note: p<0.05 for control group vs. RU486. The vertical color bar shows level of luminescence.

FIGS. 7A-B: Kaplan-Meier estimates of the overall survival of ES patients using the GR-based seven-gene signature. (A) Kaplan-Meier survival curves for the training-set patients (n=117). (B) Kaplan-Meier survival curves for the validation-set patients (n=44). Two-sided log-rank test was performed to evaluate the survival differences between the curves of the low- and high-risk groups.

FIGS. 8A-D: Specificity of interactions between steroid hormone receptors and ETS family members. (A) HEK-293T cells (6×10³) were seeded in 96-well plates. On the next day, cells were transfected with combinations of the Gluc1 plasmid encoding a fused, full length NF-κB and the Gluc2 plasmid encoding a fused GR protein. After 24 hours, cells were starved overnight for serum factors, and thereafter they were treated for 60 minutes with vehicle or with DEX (1 μM). The cells were later extracted and luminescence was determined in biological triplicates. The bar plot shows the luciferase activity in arbitrary units. ***p<0.001. (B and C) HEK-293T cells (6×10³) were seeded in 96-well plates. On the next day, cells were transfected with combinations of the Gluc1 plasmid encoding an ETS protein and a Gluc2 plasmid encoding either MR (B) or ERα (C). Twenty-four hours later, cells were starved overnight for serum factors and thereafter they were treated for 60 minutes with vehicle, DEX (1 μM) or estradiol (E2; 10 nM). Cells were then lysed and luminescence was determined. The bar plot shows the fold changes in luciferase activity induced by DEX or E2 (as compared to vehicle-treated cells) for each set of interactions between ETS family TFs and MR or ERα. Luminescence of treated cells was normalized to the control, vehicle-treated cells. *, p≤0.05. (D) HEK-293T cells were co-transfected with ERα-Gluc1 and GR-Gluc2. Twenty-four hours later, cells were treated for 60 minutes with either vehicle, DEX (1 μM) or estradiol (10 nM). Luminescence of extracted cells was determined in biological duplicates and normalized to cells treated with vehicle only. *, p≤0.05. ns, not significant.

FIGS. 9A-C: GR only moderately affects transcriptional activity of promoter-bound FLI1. HEK-293T cells (1.2×10⁴) were seeded in 48-well plates. On the next day, cells were transfected with a FLI1-BS-luciferase plasmid (2.5 μg), along with increasing amounts of either a FLI1 expression vector (A), a GR-encoding vector (B), or a combination of GR- and FLI1-plasmids (C). Luciferase activity was determined in biological triplicates 48 hours later and presented in bar plots. Basal activity was determined in cells transfected only with a FLI1-BS reporter. *, p≤0.05; ***, p≤0.001.

FIGS. 10A-C: Following DEX-induced stimulation of Ewing's sarcoma cells, GR and FLI1 translocate to the nucleus and form a physical complex, but EWS-FLI1 is constitutively nuclear. (A) A673 cells were seeded on coverslips in 6-well plates. After 24 hours, cells were starved overnight for serum factors, followed by treatment with DEX (1 μM) for the indicated time periods. Thereafter, the cells were washed in saline containing tween 20, fixed in formaldehyde (4%; in saline) and permeabilized. Prior to probing with the primary antibody (anti-GR, anti-FLI1 and anti-EWS antibodies), non-specific sites were blocked using fetal bovine serum. The incubation with the primary antibody (overnight at 4° C.) was followed by an FITC-conjugated secondary antibody and DAPI (45 minutes in dark). After three washings, cells were mounted on microscopic slides and images were captured using a Zeiss confocal microscope (40× magnification). Images were processed using the Zeiss ZEN2011 software. Quantification of nuclear localization (average±S.E.) surveyed 180 cells from each condition. Bars, 20 μm. (B) HEK-293T cells grown in 6-well plates were starved overnight for serum factors, and then treated with DEX (1 μM) for the indicated time periods. The cells were later harvested, sedimented using a centrifuge and resuspended in buffer prior to fractionation into cytoplasmic and nuclear fractions. Each fraction was resolved using gel electrophoresis followed by immunoblotting with the indicated antibodies. (C) A673 cells were probed with antibodies recognizing GR and FLI1 and processed for PLA that used a TRITC probe (red). Counterstaining used DAPI (blue) and phalloidin-FITC (green). Single antibody control experiments are shown. **P<0.01; bar, 10 μm.

FIGS. 11A-F: GR of ES cells physically associates with EWS-FLI1 and is involved in enhanced cellular migration and invasion. (A) RD-ES cells were seeded in 100-mm dishes. Once the cells reached 70% confluency, they were starved overnight for serum factors. Thereafter, cells were treated in duplicates for 60 minutes with vehicle, DEX (1 μM), or the combination of DEX and RU486 (1 μM). Thereafter, the cells were extracted and lysates were processed for co-immunoprecipitation assays using an antibody against GR and immunoblotting using an antibody specific to EWS-FLI1. The image shown is representative of three biological replicates. (B) RD-ES cells were seeded in 100-mm dishes. Once the cells reached 70% confluency, they were transfected with siRNA oligonucleotides, either control siRNAs or oligonucleotides specific to GR. Cell extracts were prepared 24 hours later and probed for GR and GAPDH. (C) Control siRNA or siRNAs specific to GR were added to RD-ES cells 24 hours prior to seeding on the upper faces of Transwell migration chambers, or Matrigel invasion chambers. Cell migration/invasion was determined 20 hours later. (D) RD-ES cells were seeded on the upper faces of migration/invasion chambers and incubated for 20 hours in full medium. DEX (1 μM) or the combination of DEX and RU486 were added to the medium and 20 hours later we fixed and stained cells that migrated to the lower face of the intervening filters. Shown are representative images of the stained cells. The bar plots show quantification (using ImageJ) of areas covered by cells. *, p≤0.05. Bar, 500 μm. (E) Migration and invasion assays of CHLA9 cells were performed as in D except for the use of a non-steroidal antagonist (D06; 10 μM) instead of RU486. *, p<0.05, ***, p<0.001. Bars, 100 μM. (F) 48-well plates were coated with Cultrex® RGF BME prior to seeding RD-ES and TC-71 cells, which were pre-transfected with Si-GR or Si—C. Unattached cells were removed 8 hours later and adherent cells were fixed with paraformaldehyde (4%), stained in crystal violet solution (0.1%) and their optical density (550 nm) was quantified in triplicates. **, p<0.01; ***, p<0.001.

FIGS. 12A-F: Inhibition of GR activity decreases growth and survival of Ewing's sarcoma cells. (A) Cell viability assays were performed using the MTT (3-(4,5-dimethylthiazol-2-yl)-2,5-diphenyltetrazolium bromide) method and three ES lines (CHLA9, A673 and RD-ES), which were treated for 24 hours with increasing concentrations of RU486. (B) Increasing concentrations of D06, a non-steroidal antagonist of GR [54], were incubated with A673 cells as in A and the MTT assay was performed in triplicates. *, p<0.05. (C) A673 cells were transfected with either FLI1-specific or control scrambled siRNAs (si-C). EWS-FLI1 knockdown efficiency was tested after 48 hours using immunoblotting. Note that the abundance of the endogenous form of FLI1 is much weaker than that of EWS-FLI1. (D) FLI1 silenced A673 cells (8000) were seeded in 96-well plates and treated with either vehicle or of RU486 (10 μM). MTT assays were performed after 48 hours of treatment. The assay was repeated twice in quadruplets. **, p<0.01, ***, p<0.001. (E) A673 cells were seeded in 100-mm dishes. Thereafter, cells were treated with DEX (1 μM), RU486 (10 or 20 μM), or the combination, for 48 hours. Shown are results of an apoptosis assay performed using an annexinV/7-AAD kit (BioLegend, Inc.). Quantification of the fractions of early and late apoptotic cells is shown in bar plots. The experiment was repeated twice. (F) The indicated ES cells were sparsely seeded in 6-well plates. Cells were later treated every other day with either vehicle, DEX (1 μM), RU486 (10 μM) or the combination. Ten days later, cells were fixed and stained with crystal violet to visualize emerging colonies. Photos are shown along with bar plots showing the quantification of colonies in 5 non-overlapping microscope fields. The experiment was repeated twice. *, p≤0.05; **, p≤0.001.

FIGS. 13A-B: A GR antagonist and a cortisol-lowering drug induce markers of cell death in animal models of Ewing's sarcoma. (A) RD-ES tumors presented in FIG. 6A were extracted and analyzed using immunoblotting and the indicated antibodies. Note that tumors from three different mice per group were analyzed. (B) Whole extracts prepared from the tumors presented in FIG. 6D were analyzed using immunoblotting and the indicated antibodies. Note that tumors from three different mice per group were analyzed.

FIGS. 14A-C: Inducible knockdown of GR expression impedes cellular migration in vitro and tumor metastasis in mice. (A) The SMARTvector Inducible Lentiviral shRNAs system specific for GR (from Dharmacon; San Diego, Calif.) was used to generate stable cell lines according to the manufacturer's protocol. Briefly, HEK-293T cells were transfected with a mixture of lentiviral packaging plasmids and two different inducible shRNAs specific for GR. Lentiviral particles were collected 48 hours after transfection. TC-71 cells were then transduced and subsequently selected for stable knockdown using puromycin (2 μg/ml). Two stable variants of TC-71 cells were selected and named inducible sh1 (iSH1) and iSH2. To inducibly knockdown GR, cells were grown for 24-72 hours in culture medium supplemented with doxycycline (1 μg/ml). GR levels were determined using immunoblotting. (B) Subclones iSH1 and iSH2 of TC-71 cells were treated for 48 hours with doxycycline (1 μg/ml) and then seeded on the upper faces of Transwell migration chambers. Following 20 hours of incubation, cells that reached the lower faces of the chambers were fixed and stained. Shown are representative images of the stained cells. The bar plots show quantification of areas covered by the cells using the ImageJ software (p≤0.05; n=2). Bars, 500 μm. (C) The two subclones of TC-71 cells (10⁶ cells), which stably express GR-specific inducible shRNAs, were injected into the tibia of 5-week old female SCID mice (10 mice per group). Once tumors reached an approximate volume of 150 mm³, mice were randomly divided into two groups: 5 mice of each group were daily treated (oral gavage) with either saline (un-induced group) or doxycycline (induced group). When a primary tumor reached 10% of body weight, both lungs were excised and analyzed for metastasis using bioluminescence imaging. Shown are whole lung images corresponding to each mouse in the end of the experiment. The vertical color bar indicates levels of luminescence.

### DESCRIPTION OF SPECIFIC EMBODIMENTS OF THE INVENTION

The present invention, in some embodiments thereof, relates to a method of treating cancer and, more particularly, but not exclusively, to cancers that are associated with expression of an oncogenic fusion protein which comprises a member of the E26 transformation-specific (ETS) family.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The cellular actions of glucocorticoids (GCs) are mediated by the glucocorticoid receptor (GR) Like other nuclear receptors, GR resides in the cytoplasm, stabilized by chaperone proteins. Once bound by GCs, GR homodimers translocate into the nucleus to regulate multiple genes, either positively or negatively.

It has previously been reported that GR maintains a crosstalk with the EGFR (epidermal growth factor receptor) pathway [12]. Upon stimulation with a ligand, EGFR activates a cascade culminating in the activation of ERK, a mitogen-activated protein kinase (MAPK). In mammary cells, this translates to cell migration, but pre-treatment with the glucocorticoid dexamethasone (DEX) strongly inhibited cell migration due to GR-mediated expression of several natural inhibitors of EGFR and the downstream MAPK pathway.

Members of the E-twenty-six (ETS) family of TFs are major downstream effectors of ERK signaling. Because GR strongly inhibits the EGFR-to-MAPK pathway, the present inventors now raised the possibility that GR physically interacts with ETS family members.

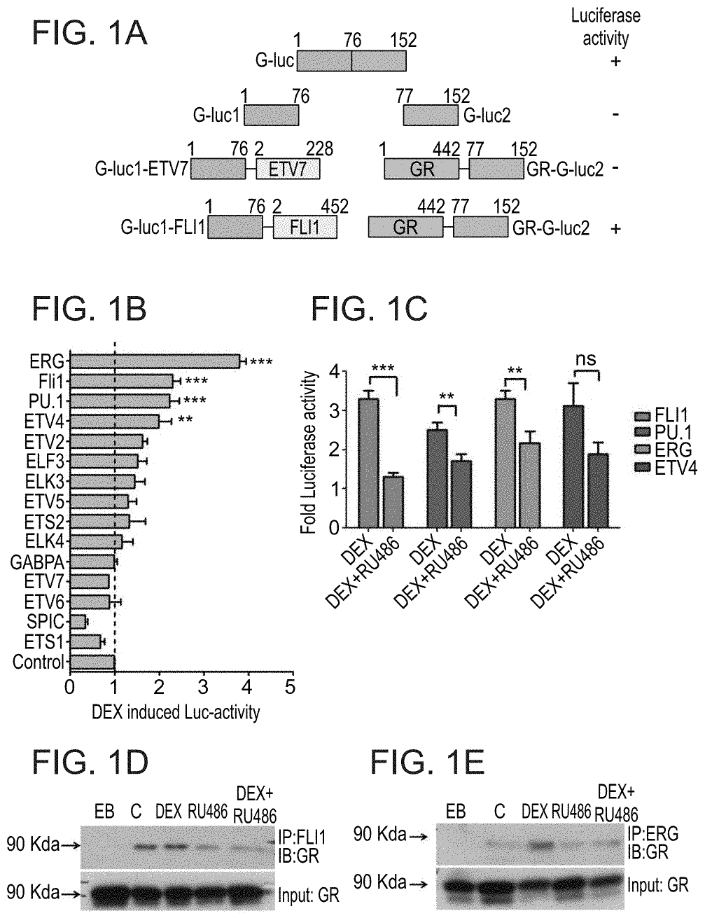

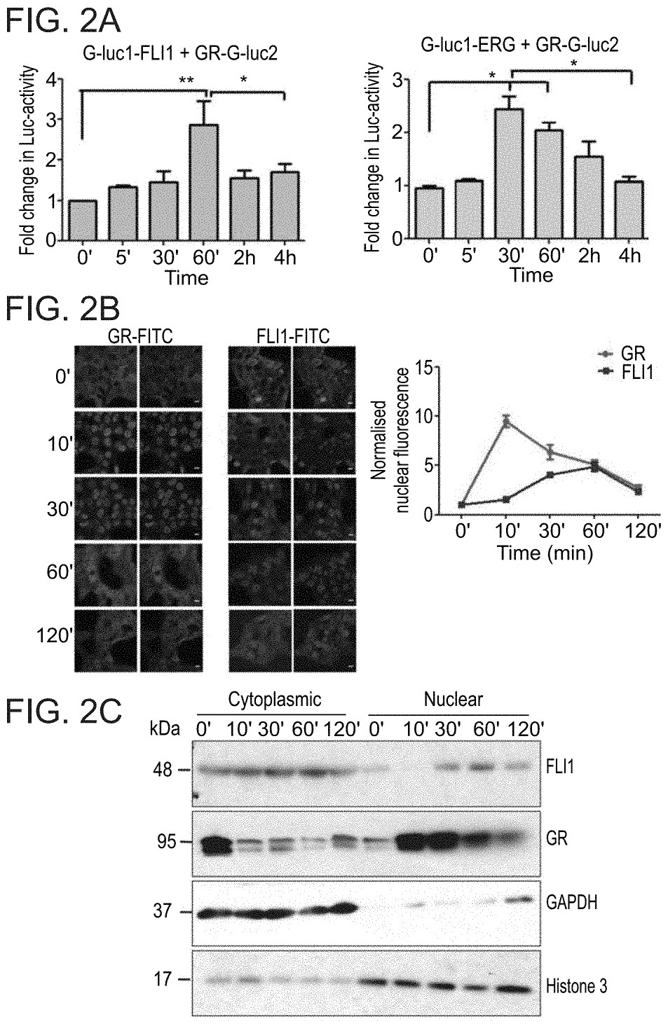

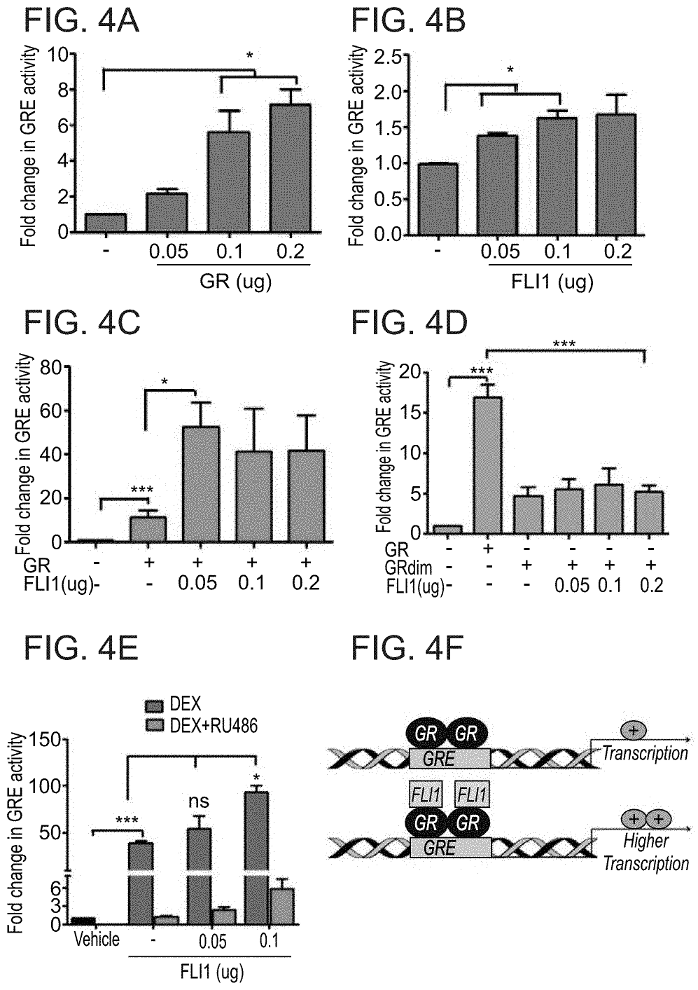

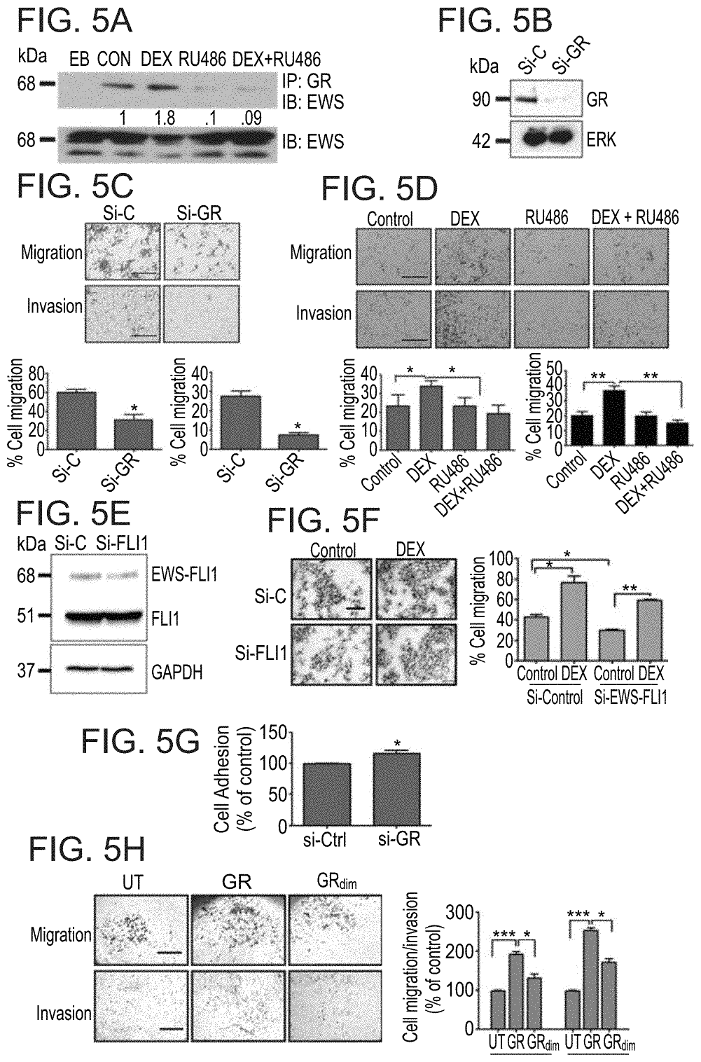

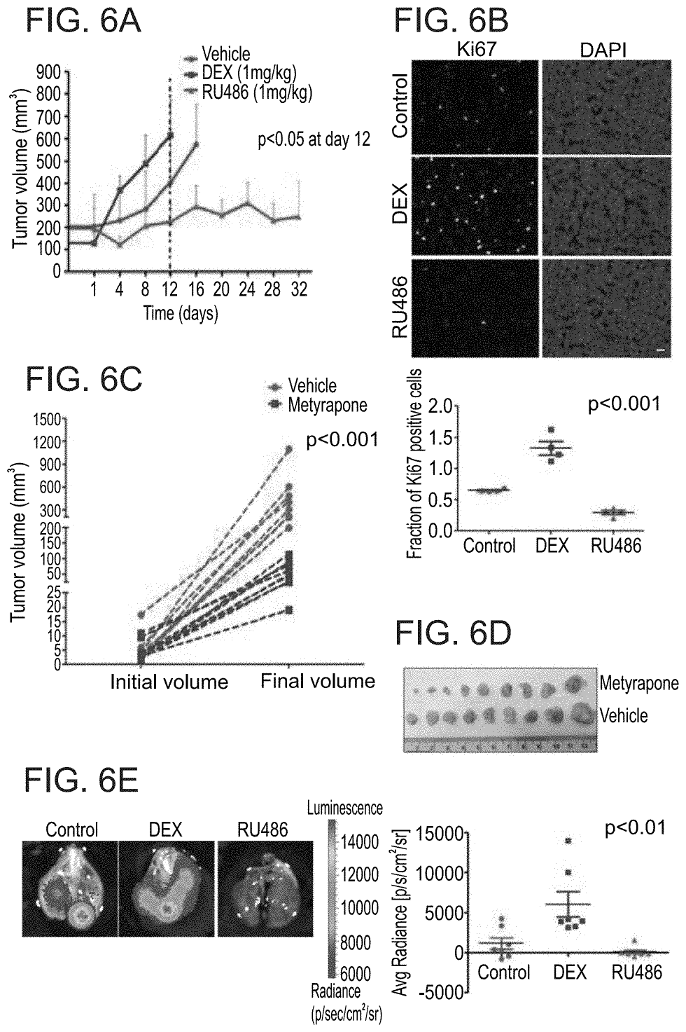

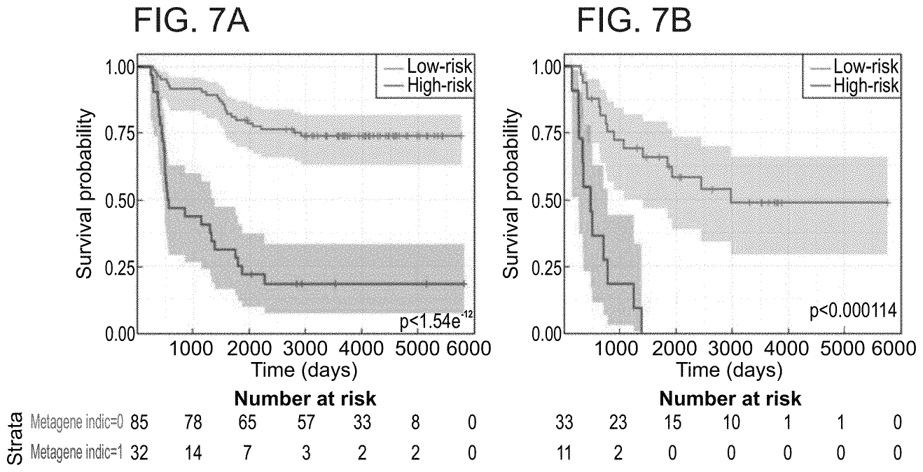

By using protein-fragment complementation assays, the present inventors found that GR recognizes FLI1 and additional ETS family TFs, which execute proliferation/migration signals (FIGS. 1A-E). Following steroid-dependent translocation of FLI1 and GR to the nucleus, the FLI1-specific (FLS) domain binds with GR's hinge and DNA-binding domains (FIGS. 2A-C). Furthermore, GR's transcriptional activity is significantly enhanced once it binds FLI1 (FIGS. 4A-F). This interaction has functional consequences in Ewing's sarcoma (ES), a childhood and adolescence bone malignancy driven by fusions between EWSR1 and FLI1: in vitro, GR knockdown weakened cellular migration and proliferation (FIGS. 5A-H) and in ES animal models antagonizing GR or lowering cortisol retarded tumor growth and metastasis (FIGS. 6A-E). In addition, a GR-based gene signature can predict very short survival of ES patients (FIGS. 7A-B). Taken together, the present findings offer a mechanistic rationale for prognosis and treatment of ES.

The present inventors further propose that agents that agents that inhibit the synthesis and/or activity of glucocorticoids can be used for the treatment of other ETS fusion-dependent cancers such as prostate cancer and hematological cancers such as leukemia.

Thus, according to a first aspect of the present invention, there is provided a method of treating a cancer selected from the group consisting of a myeloid malignancy, a lymphoid malignancy and Ewing's sarcoma in a subject in need thereof comprising administering to the subject a therapeutically effective amount of an agent that inhibits the synthesis and/or activity of cortisol, thereby treating the cancer.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the phrase “malignant disease” refers to a disease or disorder resulting from the proliferation of oncogenically transformed cells.

The term “myeloid malignancy” refers to a clonal disease of hematopoietic stem or progenitor cells. The myeloid malignancy may be chronic such as myeloproliferative neoplasms (MPN), myelodysplastic syndromes (MDS) and chronic myelomonocytic leukemia (CMML) or acute stages, i.e acute myeloid leukemia (AML). AML can occur de novo (˜80% of the cases) or follow a chronic stage (secondary AML). According to the karyotype, AMLs can be subdivided into AML with favorable, intermediate or unfavorable cytogenetic risk. MPNs comprise a variety of disorders such as chronic myeloid leukemia (CML) and non-CML MPNs such as polycythemia vera (PV), essential thrombocythemia (ET) and primary myelofibrosis (PMF).

Lymphoid malignancies include, but are not limited to Hodgkin lymphomas (HLs) and non-Hodgkin lymphomas (NHLs), plasma cell disorders, mostly represented by multiple myeloma (MM), chronic lymphocytic leukemia (CLL), and acute lymphoblastic leukemia.

The term “leukemia” refers to malignant neoplasms of the blood-forming tissues. Leukemia of the present invention includes lymphocytic (lymphoblastic) leukemia and myelogenous (myeloid or nonlymphocytic) leukemia. Exemplary types of leukemia include, but are not limited to, chronic lymphocytic leukemia, (CLL), chronic myelocytic leukemia (CML) [also known as chronic myelogenous leukemia (CML)], acute lymphoblastic leukemia (ALL) and acute myeloid leukemia (AML) [also known as acute myelogenous leukemia (AML), acute nonlymphocytic leukemia (ANLL) and acute myeloblastic leukemia (AML)]. The leukemia can be relapsed, refractory or resistant to conventional therapy.

The term “relapsed” refers to a situation where patients who have had a remission of leukemia/lymphoma after therapy have a return of leukemia/lymphoma cells in the marrow/lymph and a decrease in normal hematopoietic cells.

The term “refractory or resistant” refers to a circumstance where patients, even after intensive treatment, have residual leukemia/lymphoma cells in their marrow/lymph. The cancer may be resistant to treatment immediately or may develop a resistance during treatment.

The term “acute leukemia” means a disease that is characterized by a rapid increase in the numbers of immature blood cells that transform into malignant cells, rapid progression and accumulation of the malignant cells, which spill into the bloodstream and spread to other organs of the body.

The term “chronic leukemia” means a disease that is characterized by the excessive build-up of relatively mature, but abnormal, white blood cells.

According to one embodiment, the leukemia is an acute myeloid leukemia (AML).

The term “Ewings sarcoma” refers to a malignant small, round, blue cell tumor. It is a rare disease in which cancer cells are found in the bone or in soft tissue. The most common areas in which it occurs are the pelvis, the femur, the humerus, the ribs, the mandible and clavicle.

Agents that Inhibit the Synthesis and/or Activity of Cortisol:

1. Glucocorticoid Receptor Antagonists:

The phrase “glucocorticoid receptor” (“GR”) refers to the type II GR which specifically binds to cortisol and/or cortisol analogs such as dexamethasone (See, e.g., Turner & Muller, J Mol Endocrinol Oct. 1, 2005 35 283-292). The GR is also referred to as the cortisol receptor. The term includes isoforms of GR, recombinant GR and mutated GR. Inhibition constants (K.i) against the human GR receptor type II (Genbank: P04150) are between 0.0001 nM to 1,000 nM; preferably between 0.0005 nM to 10 nM, and most preferably between 0.001 nM to 1 nM.

The phrase “glucocorticoid receptor antagonist” refers to any agent which partially or completely inhibits (antagonizes) the binding of a glucocorticoid receptor (GR) agonist, such as cortisol, or cortisol analogs, synthetic or natural, to a GR.

A “specific glucocorticoid receptor antagonist” refers to any agent which inhibits any biological response associated with the binding of a GR to an agonist. By “specific,” the agent preferentially binds to the GR rather than other nuclear receptors, such as mineralocorticoid receptor (MR), androgen receptor (AR), or progesterone receptor (PR). It is preferred that the specific glucocorticoid receptor antagonist bind GR with an affinity that is 10 times greater ( 1/10^{th} the K_{d} value) than its affinity to the MR, AR, or PR, both the MR and PR, both the MR and AR, both the AR and PR, or to the MR, AR, and PR. In a more preferred embodiment, the specific glucocorticoid receptor antagonist binds GR with an affinity that is 100 times greater ( 1/100^{th} the K_{d} value) than its affinity to the MR, AR, or PR, both the MR and PR, both the MR and AR, both the AR and PR, or to the MR, AR, and PR.

Examples of specific glucocorticoid receptor antagonists include C113176 or C108297.

As used herein, the phrase “steroidal backbone” in the context of glucocorticoid receptor antagonists containing such refers to glucocorticoid receptor antagonists that contain modifications of the basic structure of cortisol, an endogenous steroidal glucocorticoid receptor ligand. The two most commonly known classes of structural modifications of the cortisol steroid backbone to create glucocorticoid antagonists include modifications of the 11-β-hydroxy group and modification of the 17-β-side chain (See, e.g., Lefebvre (1989) J. Steroid Biochem. 33: 557-563).

An example of a glucocorticoid receptor steroidal based antagonist is Mifepristone (RU486).

The glucocorticoid receptor antagonist may comprise a non-steroidal backbone—i.e. they do not share structural homology to, or are not modifications of, cortisol. Such compounds include synthetic mimetics and analogs of proteins, including partially peptidic, pseudopeptidic and non-peptidic molecular entities.

Non-steroidal GRA agents also include glucocorticoid receptor antagonists having a cyclohexyl-pyrimidine backbone, a fused azadecalin backbone, a heteroaryl ketone fused azadecalin backbone, or an octahydro fused azadecalin backbone. Exemplary glucocorticoid receptor antagonists having a cyclohexyl-pyrimidine backbone include those described in U.S. Pat. No. 8,685,973. Exemplary glucocorticoid receptor antagonists having a fused azadecalin backbone include those described in U.S. Pat. Nos. 7,928,237; and 8,461,172. Exemplary glucocorticoid receptor antagonists having a heteroaryl ketone fused azadecalin backbone include those described in U.S. Pat. No. 8,859,774. Exemplary glucocorticoid receptor antagonists having an octohydro fused azadecalin backbone include those described in U.S. Provisional Patent Appl. No. 61/908,333, entitled Octahydro Fused Azadecalin Glucocorticoid Receptor Modulators, Attorney Docket No. 85178-887884 (007800US), filed on Nov. 25, 2013; and U.S. Patent Application Publication No. 2015/0148341.

2. Agents that Block the Synthesis of Cortisol:

Cortisol is synthesized from cholesterol by way of pregnenolone utilizing a number of cytochrome P450 enzymes. Thus, P450 cholesterol side-chain cleavage enzyme (CYP11A) catalyzes the conversion of cholesterol to pregnenolone, which is then converted to progesterone under the influence of 3beta-hydroxysteroid dehydrogenase. Progesterone may be 17-hydroxylated to 17alpha-hydroxyprogesterone using steroid 17alpha-hydroxylase (CYP17), giving rise to cortisol by way of 11-deoxycortisol, the conversion of which to cortisol is catalyzed by steroid 11beta-hydroxylase (CYP11B1).

Several agents are known to block corticosteroid synthesis by inhibiting one or more of the enzymes involved in these pathways. Drugs inhibiting the steroidogenic pathway include mitotane, which inhibits the effects of corticotrophin and possibly 11beta-hydroxylase, aminoglutethimide, which inhibits cholesterol side-chain cleavage enzyme and is also an aromatase inhibitor, metyrapone, which inhibits steroid 11beta-hydroxylase, and trilostane, which inhibits 3beta-hydroxysteroid dehydrogenase. The antifungal agent ketoconazole is also a potent inhibitor of steroid biosynthesis. It blocks steroid 11beta-hydroxylase and also, more potently, the conversion of 17alpha-hydroxyprogesterone to androstenedione under the influence of 17alpha-hydroxylase Weber et al (1993). Etomidate, an anesthetic agent, is a potent inhibitor of 11beta-hydroxylase Dorr et al (1984).

According to a particular embodiment, the agent that inhibits cortisol synthesis is selected from the group consisting of metyrapone ketoconazole, levoketoconazole, LCI699, mitotane, aminoglutethimide and etomidate.

According to a particular embodiment, the agent is metyrapone.

The present inventors further contemplate polynucleotide agents that are capable of down-regulating expression of a component of the cortisol synthesis pathway. Such agents include siRNA agents, antisense agents, CRISPR agents, ribozyme agents etc. Each of these agents rely on specificity due to the hybridization of same to the gene encoding the component of the cortisol pathway or the RNA transcribed therefrom.

The subject may be a healthy animal or human subject undergoing a routine well-being check up. Alternatively, the subject may be at risk of having cancer (e.g., a genetically predisposed subject, a subject with medical and/or family history of cancer, a subject who has been exposed to carcinogens, occupational hazard, environmental hazard] and/or a subject who exhibits suspicious clinical signs of a malignant disease [e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness).

As used herein, the phrase “malignant disease” refers to a disease or disorder resulting from the proliferation of oncogenically transformed cells.

In one embodiment, the subject that is treated expresses a genomic ETS rearrangement.

Thus, according to another aspect of the present invention there is provided a method of treating a subject having a cancer characterized by expression of a fusion protein which comprises a member of the E-twenty-six (ETS) family, the method comprising:

(a) analyzing in a sample of the subject for the presence of a genomic ETS rearrangement; and

(b) administering to the subject a therapeutically effective amount of an agent that inhibits the synthesis and/or activity of cortisol, thereby treating the cancer.

Examples of cancer characterized by the expression of a fusion protein which comprises a member of the E-twenty-six (ETS) family include hematological cancers (such as leukemia), prostate cancer and Ewing's sarcoma.

As used herein, “prostate cancer” is defined as cancer of the prostate gland, typically adenocarcinoma of the prostate gland.

The E-twenty-six (ETS) family of transcription factors regulate the intra-cellular signaling pathways controlling gene expression. As downstream effectors, they activate or repress specific target genes. As upstream effectors, they are responsible for the spatial and temporal expression of numerous growth factor receptors. Almost 30 members of this family have been identified and implicated in a wide range of physiological and pathological processes.

In one embodiment, the ETS family member includes, but is not limited to: ERG; ETV1 (ER81); FLI1; ETS1; ETS2; ELK1; ETV7 (TEL2); GABPa; ELF1; ETV4 (E1AF; PEA3); ETV5 (ERM); ERF; PEA3/E1AF; PU.1; ESE1/ESX; SAP1 (ELK4); ETV3 (METS); EWS/FLI1; ESE1; ESE2 (ELF5); ESE3; PDEF; NET (ELK3; SAP2); and NERF (ELF2).

According to a specific embodiment, the ETS family member is not FEV or ETV6.

According to a particular embodiment, the ETS family member is ERG.

ERG (NM_004449), in particular, has been demonstrated to be highly expressed in prostate epithelium relative to other normal human tissues. The ERG gene is located on chromosome 21. The gene is located at 38,675,671-38,955,488 base pairs from the pter. The ERG gene is 279,817 total bp; minus strand orientation. The corresponding ERG cDNA and protein sequences are given at GenBank accession no. M17254 and GenBank accession no. NPO4440 (Swiss Protein acc. no. P11308), respectively.

According to another embodiment, the ETS family member is ETV1. The ETV1 gene is located on chromosome 7 (GenBank accession nos. NC_000007.11; NC_086703.11; and NT_007819.15). The gene is located at 13,708330-13,803,555 base pairs from the pter. The ETV1 gene is 95,225 bp total, minus strand orientation. The corresponding ETV1 cDNA and protein sequences are given at GenBank accession no. NM_004956 and GenBank accession no. NP_004947 (Swiss protein acc. no. P50549), respectively.

According to another embodiment, the ETS family member is ETV4. The human ETV4 gene is located on chromosome 14 (GenBank accession nos. NC_000017.9; NT_010783.14; and NT_086880.1). The gene is at 38,960,740-38,979,228 base pairs from the pter. The ETV4 gene is 18,488 bp total, minus strand orientation. The corresponding ETV4 cDNA and protein sequences are given at GenBank accession no. NM_001986 and GenBank accession no. NP_01977 (Swiss protein acc. no. P43268), respectively.

The term “genomic ETS rearrangement” refers to a changes in the genetic linkage relationship of discrete chromosomal fragments, involving deletions, duplications, insertions, inversions or translocations of the ETS gene or part thereof.

In one embodiment, the genomic ETS rearrangement generates a fusion protein which comprises the member of the E-twenty-six (ETS) family.

The fusion of an ETS family member gene to a second gene is detectable as DNA, RNA or protein. Typically, a cellular sample is retrieved from the subject in order to confirm/ascertain that he/she expresses the fusion protein.

In one embodiment, the sample is a fluid sample, including, but not limited to whole blood, plasma, serum and urine.

In another embodiment, the sample is a tissue sample (e.g. a tissue biopsy).

In still another embodiment, the sample is a bone marrow sample.

In one embodiment, the gene fusion is detectable as a chromosomal rearrangement of genomic DNA having a 5′ portion of the ETS family member gene and a 3′ portion of the second gene. In another embodiment, the gene fusion is detectable as a chromosomal rearrangement of genomic DNA having a 3′ portion of the ETS family member gene and a 5′ portion of the second gene.

Once transcribed, the gene fusion is detectable as a chimeric mRNA having a 5′ portion from the second gene and a 3′ portion from the ETS family member gene. Alternatively, the gene fusion is detectable as a chimeric mRNA having a 3′ portion from the second gene and a 5′ portion from the ETS family member gene.

Once translated, the gene fusion is detectable as an amino-terminally, or carboxy-terminally truncated ETS family member protein resulting from the fusion of the second gene to the ETS family member gene. The truncated ETS family member protein may differ from their respective native proteins in amino acid sequence, post-translational processing and/or secondary, tertiary or quaternary structure. Such differences, if present, can be used to identify the presence of the gene fusion. Specific methods of detection are described in more detail below.

Certain gene fusions are more common than others in prostate cancer. Examples of such include gene fusions of TMPRSS2 with ERG, ETV1, ETV4, or FLI1.

Examples of ETS gene fusions in hematological disorders include TLS/ERG (Shimizu et al., Proc. Natl. Acad. Sci. USA, 90: 10280-10284, 1993) and ETV6/MN1 (Buijs et al., Oncogene, 10: 1511-1519, 1995) and ETV6/RUNX1.

Examples of ETS gene fusions in Ewing's Sarcoma include EWS-FLI1 and EWS-ERG (Sorensen et al., Nat. Genet., 6: 146-151, 1994).

Methods of analyzing for the presence of ETS fusion proteins are detailed herein below.

1. Chromosomal and DNA Staining Methods:

FISH—Methods of employing FISH analysis on interphase chromosomes are known in the art. Briefly, directly-labeled probes [e.g., the CEP X green and Y orange (Abbott cat no. 5J10-51)] are mixed with hybridization buffer (e.g., LSI/WCP, Abbott) and a carrier DNA (e.g., human Cot 1 DNA, available from Abbott). The probe solution is applied on microscopic slides containing e.g., transcervical cytospin specimens and the slides are covered using a coverslip. The probe-containing slides are denatured for 3 minutes at 70° C. and are further incubated for 48 hours at 37° C. using an hybridization apparatus (e.g., HYBrite, Abbott Cat. No. 2J11-04). Following hybridization, the slides are washed for 2 minutes at 72° C. in a solution of 0.3% NP-40 (Abbott) in 60 mM NaCl and 6 mM NaCitrate (0.4×SSC). Slides are then immersed for 1 minute in a solution of 0.1% NP-40 in 2×SSC at room temperature, following which the slides are allowed to dry in the dark. Counterstaining is performed using, for example, DAPI II counterstain (Abbott).

PRINS analysis has been employed in the detection of gene deletion (Tharapel S A and Kadandale J S, 2002. Am. J. Med. Genet. 107: 123-126), determination of fetal sex (Orsetti, B., et al., 1998. Prenat. Diagn. 18: 1014-1022), and identification of chromosomal aneuploidy (Mennicke, K. et al., 2003. Fetal Diagn. Ther. 18: 114-121).

Methods of performing PRINS analysis are known in the art and include for example, those described in Coullin, P. et al. (Am. J. Med. Genet. 2002, 107: 127-135); Findlay, I., et al. (J. Assist. Reprod. Genet. 1998, 15: 258-265); Musio, A., et al. (Genome 1998, 41: 739-741); Mennicke, K., et al. (Fetal Diagn. Ther. 2003, 18: 114-121); Orsetti, B., et al. (Prenat. Diagn. 1998, 18: 1014-1022). Briefly, slides containing interphase chromosomes are denatured for 2 minutes at 71° C. in a solution of 70% formamide in 2×SSC (pH 7.2), dehydrated in an ethanol series (70, 80, 90 and 100%) and are placed on a flat plate block of a programmable temperature cycler (such as the PTC-200 thermal cycler adapted for glass slides which is available from MJ Research, Waltham, Mass., USA). The PRINS reaction is usually performed in the presence of unlabeled primers and a mixture of dNTPs with a labeled dUTP (e.g., fluorescein-12-dUTP or digoxigenin-11-dUTP for a direct or indirect detection, respectively). Alternatively, or additionally, the sequence-specific primers can be labeled at the 5′ end using e.g., 1-3 fluorescein or cyanine 3 (Cy3) molecules. Thus, a typical PRINS reaction mixture includes sequence-specific primers (50-200 pmol in a 50 μl reaction volume), unlabeled dNTPs (0.1 mM of dATP, dCTP, dGTP and 0.002 mM of dTTP), labeled dUTP (0.025 mM) and Taq DNA polymerase (2 units) with the appropriate reaction buffer. Once the slide reaches the desired annealing temperature the reaction mixture is applied on the slide and the slide is covered using a cover slip. Annealing of the sequence-specific primers is allowed to occur for 15 minutes, following which the primed chains are elongated at 72° C. for another 15 minutes. Following elongation, the slides are washed three times at room temperature in a solution of 4×SSC/0.5% Tween-20 (4 minutes each), followed by a 4-minute wash at PBS. Slides are then subjected to nuclei counterstain using DAPI or propidium iodide. The fluorescently stained slides can be viewed using a fluorescent microscope and the appropriate combination of filters (e.g., DAPI, FITC, TRITC, FITC-rhodamin).

It will be appreciated that several primers which are specific for several targets can be used on the same PRINS run using different 5′ conjugates. Thus, the PRINS analysis can be used as a multicolor assay for the determination of the presence, and/or location of several genes or chromosomal loci.

In addition, as described in Coullin et al., (2002, Supra) the PRINS analysis can be performed on the same slide as the FISH analysis, preferably, prior to FISH analysis.

High-resolution multicolor banding (MCB) on interphase chromosomes—This method, which is described in detail by Lemke et al. (Am. J. Hum. Genet. 71: 1051-1059, 2002), uses YAC/BAC and region-specific microdissection DNA libraries as DNA probes for interphase chromosomes. Briefly, for each region-specific DNA library 8-10 chromosome fragments are excised using microdissection and the DNA is amplified using a degenerated oligonucleotide PCR reaction. For example, for MCB staining of chromosome 5, seven overlapping microdissection DNA libraries were constructed, two within the p arm and five within the q arm (Chudoba I., et al., 1999; Cytogenet. Cell Genet. 84: 156-160). Each of the DNA libraries is labeled with a unique combination of fluorochromes and hybridization and post-hybridization washes are carried out using standard protocols (see for example, Senger et al., 1993; Cytogenet. Cell Genet. 64: 49-53). Analysis of the multicolor-banding can be performed using the isis/mFISH imaging system (MetaSystems GmbH, Altlussheim, Germany). It will be appreciated that although MCB staining on interphase chromosomes was documented for a single chromosome at a time, it is conceivable that additional probes and unique combinations of fluorochromes can be used for MCB staining of two or more chromosomes at a single MCB analysis. Thus, this technique can be used along with some embodiments of the invention to identify fetal chromosomal aberrations, particularly, for the detection of specific chromosomal abnormalities which are known to be present in other family members.

Quantitative FISH (Q-FISH)—In this method chromosomal abnormalities are detected by measuring variations in fluorescence intensity of specific probes. Q-FISH can be performed using Peptide Nucleic Acid (PNA) oligonucleotide probes. PNA probes are synthetic DNA mimics in which the sugar phosphate backbone is replaced by repeating N-(2-aminoethyl) glycine units linked by an amine bond and to which the nucleobases are fixed (Pellestor F and Paulasova P, 2004; Chromosoma 112: 375-380). Thus, the hydrophobic and neutral backbone enables high affinity and specific hybridization of the PNA probes to their nucleic acid counterparts (e.g., chromosomal DNA). Such probes have been applied on interphase nuclei to monitor telomere stability (Slijepcevic, P. 1998; Mutat. Res. 404:215-220; Henderson S., et al., 1996; J. Cell Biol. 134: 1-12), the presence of Fanconi aneamia (Hanson H, et al., 2001, Cytogenet. Cell Genet. 93: 203-6) and numerical chromosome abnormalities such as trisomy 18 (Chen C, et al., 2000, Mamm. Genome 10: 13-18), as well as monosomy, duplication, and deletion (Taneja K L, et al., 2001, Genes Chromosomes Cancer. 30: 57-63).

Alternatively, Q-FISH can be performed by co-hybridizing whole chromosome painting probes (e.g., for chromosomes 21 and 22) on interphase nuclei as described in Truong K et al, 2003, Prenat. Diagn. 23: 146-51.

2. Analysis of Sequence Alterations at the DNA Level:

To determine sequence alterations in the ETS gene, DNA is first obtained from a biological sample of the tested subject. Such biological samples include, but are not limited to, body fluids such as whole blood, serum, plasma, cerebrospinal fluid, urine, lymph fluids, and various external secretions of the respiratory, intestinal and genitourinary tracts, tears, saliva, milk as well as white blood cells, malignant tissues, amniotic fluid and chorionic villi.

Once the sample is obtained, DNA is extracted using methods which are well known in the art, involving tissue mincing, cell lysis, protein extraction and DNA precipitation using 2 to 3 volumes of 100% ethanol, rinsing in 70% ethanol, pelleting, drying and resuspension in water or any other suitable buffer (e.g., Tris-EDTA). Preferably, following such procedure, DNA concentration is determined such as by measuring the optical density (OD) of the sample at 260 nm (wherein 1 unit OD=50 μg/ml DNA).

To determine the presence of proteins in the DNA solution, the OD 260/OD 280 ratio is determined. Preferably, only DNA preparations having an OD 260/OD 280 ratio between 1.8 and 2 are used in the following procedures described hereinbelow.

The sequence alteration (or SNP) of some embodiments of the invention can be identified using a variety of methods. One option is to determine the entire gene sequence of a PCR reaction product (see sequence analysis, hereinbelow). Alternatively, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): This method uses a change in a single nucleotide (the SNP nucleotide) which modifies a recognition site for a restriction enzyme resulting in the creation or destruction of an RFLP. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

The DNA sample is preferably amplified prior to determining sequence alterations, since many genotyping methods require amplification of the DNA region carrying the sequence alteration of interest.

In any case, once DNA is obtained, determining the presence of a sequence alteration in the ETS gene is effected using methods which typically involve the use of oligonucleotides which specifically hybridize with the nucleic acid sequence alterations in the ETS gene, such as those described hereinabove.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions.

Oligonucleotides designed according to the teachings of some embodiments of the invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotide of some embodiments of the invention is of at least 17, at least 18, at least 19, at least 20, at least 22, at least 25, at least 30 or at least 40, bases specifically hybridizable with sequence alterations described hereinabove.

The oligonucleotides of some embodiments of the invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder.

Specific examples of preferred oligonucleotides useful according to some embodiments of the invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to some embodiments of the invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in some embodiments of the invention are disclosed in U.S. Pat. No. 6,303,374.

Oligonucleotides of some embodiments of the invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications. Still further base substitutions include the non-standard bases disclosed in U.S. Pat. Nos. 8,586,303, 8,614,072, 8,871,469 and 9,062,336, all to Benner et al: for example, the non-standard dZ:dP nucleobase pair which Benner et al has shown can be incorporated into DNA by DNA polymerases to yield amplicons with multiple non-standard nucleotides.

Preferred methods of detecting sequence alterations involve directly determining the identity of the nucleotide at the alteration site by a sequencing assay, an enzyme-based mismatch detection assay, or a hybridization assay. The following is a description of some preferred methods which can be utilized by some embodiments of the invention.

Sequencing analysis—The isolated DNA is subjected to automated dideoxy terminator sequencing reactions using a dye-terminator (unlabeled primer and labeled di-deoxy nucleotides) or a dye-primer (labeled primers and unlabeled di-deoxy nucleotides) cycle sequencing protocols. For the dye-terminator reaction, a PCR reaction is performed using unlabeled PCR primers followed by a sequencing reaction in the presence of one of the primers, deoxynucleotides and labeled di-deoxy nucleotide mix. For the dye-primer reaction, a PCR reaction is performed using PCR primers conjugated to a universal or reverse primers (one at each direction) followed by a sequencing reaction in the presence of four separate mixes (correspond to the A, G, C, T nucleotides) each containing a labeled primer specific the universal or reverse sequence and the corresponding unlabeled di-deoxy nucleotides.

Microsequencing analysis—This analysis can be effected by conducting microsequencing reactions on specific regions of the ETS gene which may be obtained by amplification reaction (PCR) such as mentioned hereinabove. Genomic or cDNA amplification products are then subjected to automated microsequencing reactions using ddNTPs (specific fluorescence for each ddNTP) and an appropriate oligonucleotide microsequencing primer which can hybridize just upstream of the alteration site of interest. Once specifically extended at the 3′ end by a DNA polymerase using a complementary fluorescent dideoxynucleotide analog (thermal cycling), the primer is precipitated to remove the unincorporated fluorescent ddNTPs. The reaction products in which fluorescent ddNTPs have been incorporated are then analyzed by electrophoresis on sequencing machines (e.g., ABI 377) to determine the identity of the incorporated base, thereby identifying the sequence alteration in the ETS gene of some embodiments of the invention.

It will be appreciated that the extended primer may also be analyzed by MALDI-TOF Mass Spectrometry. In this case, the base at the alteration site is identified by the mass added onto the microsequencing primer [see Haff and Smirnov, (1997) Nucleic Acids Res. 25(18):3749-50].

Solid phase microsequencing reactions which have been recently developed can be utilized as an alternative to the microsequencing approach described above. Solid phase microsequencing reactions employ oligonucleotide microsequencing primers or PCR-amplified products of the DNA fragment of interest which are immobilized. Immobilization can be carried out, for example, via an interaction between biotinylated DNA and streptavidin-coated microtitration wells or avidin-coated polystyrene particles.

In such solid phase microsequencing reactions, incorporated ddNTPs can either be radiolabeled [see Syvanen, (1994),] Clin Chim Acta 1994; 226(2):225-236] or linked to fluorescein (see Livak and Hainer, (1994) Hum Mutat 1994; 3(4):379-385]. The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such asp-nitrophenyl phosphate).

Other reporter-detection conjugates include: ddNTP linked to dinitrophenyl (DNP) and anti-DNP alkaline phosphatase conjugate [see Harju et al., (1993) Clin Chem 39:2282-2287]; and biotinylated ddNTP and horseradish peroxidase-conjugated streptavidin with o-phenylenediamine as a substrate (see WO 92/15712).

A diagnostic kit based on fluorescein-linked ddNTP with antifluorescein antibody conjugated with alkaline phosphatase is commercially available from GamidaGen Ltd (PRONTO).

Other modifications of the microsequencing protocol are described by Nyren et al. (1993) Anal Biochem 208(1):171-175 and Pastinen et al. (1997) Genome Research 7:606-614.

Mismatch detection assays based on polymerases and ligases—The “Oligonucleotide Ligation Assay” (OLA) uses two oligonucleotides which are designed to be capable of hybridizing to abutting sequences of a single strand of a target molecules. One of the oligonucleotides is biotinylated, and the other is detectably labeled. If the precise complementary sequence is found in a target molecule, the oligonucleotides will hybridize such that their termini abut, and create a ligation substrate that can be captured and detected. OLA is capable of detecting single nucleotide polymorphisms and may be advantageously combined with PCR as described by Nickerson et al. (1990) Proc. Natl. Acad. Sci. U.S.A. 87:8923-8927. In this method, PCR is used to achieve the exponential amplification of target DNA, which is then detected using OLA.

Ligase/Polymerase-mediated Genetic Bit Analysis™ is another method for determining the identity of a particular sequence in a nucleic acid molecule (WO 95/21271). This method involves the incorporation of a nucleoside triphosphate that is complementary to the nucleotide present at the preselected site onto the terminus of a primer molecule, and their subsequent ligation to a second oligonucleotide. The reaction is monitored by detecting a specific label attached to the reaction's solid phase or by detection in solution.

Hybridization Assay Methods—Hybridization based assays which allow the detection of a specific sequence rely on the use of oligonucleotide which can be 10, 15, 20, or 30 to 100 nucleotides long preferably from 10 to 50, more preferably from 40 to 50 nucleotides.

By way of example, hybridization of short nucleic acids (below 200 bp in length, e.g. 17-40 bp in length) can be effected by the following hybridization protocols depending on the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tm, final wash solution of 3 M TMACI, 0.01 M sodium phosphate (pH 6.8), 1 mM EDTA (pH 7.6), 0.5% SDS at 1-1.5° C. below the Tm; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tm, final wash solution of 3 M TMACI, 0.01 M sodium phosphate (pH 6.8), 1 mM EDTA (pH 7.6), 0.5% SDS at 1-1.5° C. below the Tm, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample (target). For example, oligonucleotides of some embodiments of the invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, FluorX (Amersham) and others [e.g., Kricka et al. (1992), Academic Press San Diego, Calif] can be attached to the oligonucleotides.

Traditional hybridization assays include PCR, RT-PCR, RNase protection, in-situ hybridization, primer extension, Southern blot, Northern Blot and dot blot analysis.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

Two recently developed assays allow hybridization-based allele discrimination with no need for separations or washes [see Landegren U. et al., (1998) Genome Research, 8:769-776]. The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. C1 cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time [see Livak et al., 1995 Hum Mutat 3(4):379-385]. In an alternative homogeneous hybridization based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., (1998) Nature Biotechnology. 16:49].

It will be appreciated that a variety of controls may be usefully employed to improve accuracy of hybridization assays. For instance, samples may be hybridized to an irrelevant probe and treated with RNAse A prior to hybridization, to assess false hybridization.

U.S. Pat. No. 5,451,503 provides several examples of oligonucleotide configurations which can be utilized to detect SNPs in template DNA or RNA.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989; Orita et al. 1989, Proc. Natl. Acad. Sci. U.S.A. 86:2776-2770).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

Pyrosequencing™ analysis (Pyrosequencing, Inc. Westborough, Mass., USA): This technique is based on the hybridization of a sequencing primer to a single stranded, PCR-amplified, DNA template in the presence of DNA polymerase, ATP sulfurylase, luciferase and apyrase enzymes and the adenosine 5′ phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated.

Acycloprime™ analysis (Perkin Elmer, Boston, Mass., USA): This technique is based on fluorescent polarization (FP) detection. Following PCR amplification of the sequence containing the SNP of interest, excess primer and dNTPs are removed through incubation with shrimp alkaline phosphatase (SAP) and exonuclease I. Once the enzymes are heat inactivated, the Acycloprime-FP process uses a thermostable polymerase to add one of two fluorescent terminators to a primer that ends immediately upstream of the SNP site. The terminator(s) added are identified by their increased FP and represent the allele(s) present in the original DNA sample. The Acycloprime process uses AcycloPol™, a novel mutant thermostable polymerase from the Archeon family, and a pair of AcycloTerminators™ labeled with R110 and TAMRA, representing the possible alleles for the SNP of interest. AcycloTerminator™ non-nucleotide analogs are biologically active with a variety of DNA polymerases. Similarly to 2′, 3′-dideoxynucleotide-5′-triphosphates, the acyclic analogs function as chain terminators. The analog is incorporated by the DNA polymerase in a base-specific manner onto the 3′-end of the DNA chain, and since there is no 3′-hydroxyl, is unable to function in further chain elongation. It has been found that AcycloPol has a higher affinity and specificity for derivatized AcycloTerminators than various Taq mutant have for derivatized 2′,3′-dideoxynucleotide terminators.

Reverse dot blot: This technique uses labeled sequence specific oligonucleotide probes and unlabeled nucleic acid samples. Activated primary amine-conjugated oligonucleotides are covalently attached to carboxylated nylon membranes. After hybridization and washing, the labeled probe, or a labeled fragment of the probe, can be released using oligomer restriction, i.e., the digestion of the duplex hybrid with a restriction enzyme. Circular spots or lines are visualized colorimetrically after hybridization through the use of streptavidin horseradish peroxidase incubation followed by development using tetramethylbenzidine and hydrogen peroxide, or via chemiluminescence after incubation with avidin alkaline phosphatase conjugate and a luminous substrate susceptible to enzyme activation, such as CSPD, followed by exposure to x-ray film.

It will be appreciated that advances in the field of SNP detection have provided additional accurate, easy, and inexpensive large-scale SNP genotyping techniques, such as dynamic allele-specific hybridization (DASH, Howell, W. M. et al., 1999. Dynamic allele-specific hybridization (DASH). Nat. Biotechnol. 17: 87-8), microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5], the TaqMan system (Holland, P. M. et al., 1991. Detection of specific polymerase chain reaction product by utilizing the 5′→3′ exonuclease activity of Thermus aquaticus DNA polymerase. Proc Natl Acad Sci USA. 88: 7276-80), as well as various DNA “chip” technologies such as the GeneChip microarrays (e.g., Affymetrix SNP chips) which are disclosed in U.S. Pat. No. 6,300,063 to Lipshutz, et al. 2001, which is fully incorporated herein by reference, Genetic Bit Analysis (GBA™) which is described by Goelet, P. et al. (PCT Appl. No. 92/15712), peptide nucleic acid (PNA, Ren B, et al., 2004. Nucleic Acids Res. 32: e42) and locked nucleic acids (LNA, Latorra D, et al., 2003. Hum. Mutat. 22: 79-85) probes, Molecular Beacons (Abravaya K, et al., 2003. Clin Chem Lab Med. 41: 468-74), intercalating dye [Germer, S. and Higuchi, R. Single-tube genotyping without oligonucleotide probes. Genome Res. 9:72-78 (1999)], FRET primers (Solinas A et al., 2001. Nucleic Acids Res. 29: E96), AlphaScreen (Beaudet L, et al., Genome Res. 2001, 11(4): 600-8), SNPstream (Bell P A, et al., 2002. Biotechniques. Suppl.: 70-2, 74, 76-7), Multiplex minisequencing (Curcio M, et al., 2002. Electrophoresis. 23: 1467-72), SnaPshot (Turner D, et al., 2002. Hum Immunol. 63: 508-13), MassEXTEND (Cashman J R, et al., 2001. Drug Metab Dispos. 29: 1629-37), GOOD assay (Sauer S, and Gut I G. 2003. Rapid Commun. Mass. Spectrom. 17: 1265-72), Microarray minisequencing (Liljedahl U, et al., 2003. Pharmacogenetics. 13: 7-17), arrayed primer extension (APEX) (Tonisson N, et al., 2000. Clin. Chem. Lab. Med. 38: 165-70), Microarray primer extension (O'Meara D, et al., 2002. Nucleic Acids Res. 30: e75), Tag arrays (Fan J B, et al., 2000. Genome Res. 10: 853-60), Template-directed incorporation (TDI) (Akula N, et al., 2002. Biotechniques. 32: 1072-8), fluorescence polarization (Hsu T M, et al., 2001. Biotechniques. 31: 560, 562, 564-8), Colorimetric oligonucleotide ligation assay (OLA, Nickerson D A, et al., 1990. Proc. Natl. Acad. Sci. USA. 87: 8923-7), Sequence-coded OLA (Gasparini P, et al., 1999. J. Med. Screen. 6: 67-9), Microarray ligation, Ligase chain reaction, Padlock probes, Rolling circle amplification, Invader assay (reviewed in Shi M M. 2001. Enabling large-scale pharmacogenetic studies by high-throughput mutation detection and genotyping technologies. Clin Chem. 47: 164-72), coded microspheres (Rao K V et al., 2003. Nucleic Acids Res. 31: e66) MassArray (Leushner J, Chiu N H, 2000. Mol Diagn. 5: 341-80), heteroduplex analysis, mismatch cleavage detection, and other conventional techniques as described in Sheffield et al. (1991), White et al. (1992), Grompe et al. (1989 and 1993), exonuclease-resistant nucleotide derivative (U.S. Pat. No. 4,656,127).

3. Methods of Detecting Sequence Alteration at the RNA Level

Alteration in the sequence of RNA can be determined using methods known in the arts.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

DNA Microarrays/DNA Chips:

The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W. H. Freeman, New York. (2000)].

4. Sequence Alterations at the Protein Level:

Sequence alterations can also be determined at the protein level. While chromatography and electrophoretic methods are preferably used to detect large variations in molecular weight, such as detection of the truncated ETS protein, immunodetection assays such as ELISA and western blot analysis, immunohistochemistry and the like, which may be effected using antibodies specific to smaller sequence alterations are preferably used to detect point mutations and subtle changes in molecular weight.

Thus, the invention according to some embodiments thereof also envisages the use of serum immunoglobulins, polyclonal antibodies or fragments thereof, (i.e., immunoreactive derivatives thereof), or monoclonal antibodies or fragments thereof. Monoclonal antibodies or purified fragments of the monoclonal antibodies having at least a portion of an antigen-binding region, including the fragments described hereinbelow, chimeric or humanized antibodies and complementarily determining regions (CDR).

Exemplary methods for analyzing protein alterations are set forth herein below.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

Once the subject is predicted to respond to an agent that inhibits the synthesis and/or activity of cortisol and is diagnosed as expressing the ETS fusion protein, it is advisable to treat the subject accordingly.

If the ETS fusion protein has been established as being absent in the sample of the subject, then the present inventors contemplate not treating the subject with an agent that inhibits the synthesis and/or activity of cortisol and seeking alternative treatments, such as anti-cancer agents known to be therapeutic for that cancer.

The agent that inhibits the synthesis and/or activity of cortisol of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent that inhibits the synthesis and/or activity of cortisol which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient that are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Therapeutic regimen for treatment of cancer suitable for combination with the agent that inhibits the synthesis and/or activity of cortisol include, but are not limited to chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy.

Examples of anti-cancer drugs that can be co-administered (or even co-formulated) with the agents that agent that inhibit the synthesis and/or activity of cortisol include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

The present inventors have further shown that Ewing's sarcoma tumor cells expressing a particular signature can be used to predict survival of the patient.

Thus, according to another aspect of the present invention, there is provided a method of predicting survival of a subject having a Ewing's sarcoma comprising analyzing in a sample of the subject for the presence of cells having a signature comprising each of PDIA6, COL6A3, TMED10, SEC61G, PPA1, IGFBP7, and RPL37A, wherein the signature is indicative of short survival.

Methods of analyzing for the expression of the above described genes can be effected at the RNA level or the protein level as further described herein above.

According to some embodiments of the invention, the method further comprises informing the subject of the predicted prognosis of the subject.

As used herein the phrase “informing the subject” refers to advising the subject that based on the expression profile of the cancer cells, the subject should seek a suitable treatment regimen. Once the prognosis is determined, the results can be recorded in the subject's medical file, which may assist in selecting a treatment regimen and/or determining prognosis of the subject.

According to some embodiments of the invention, the method further comprising recording the results of the subject to expression profile of the cancer cells in the subject's medical file.

As mentioned, the prediction of the prognosis of a subject can be used to select the treatment regimen of a subject and thereby treat the subject in need thereof.

It is expected that during the life of a patent maturing from this application many relevant agents that inhibit the synthesis and/or activity of cortisol will be developed and the scope of the phrase “agents that inhibit the synthesis and/or activity of cortisol” is intended to include all such new technologies a priori.]

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Cell lines and materials: CHLA9 and TC-71 cell lines were from the Children's Cancer Research Institute, (Vienna) and Georgetown University (Dr. Jeffrey Toretsky), respectively. Other lines were from the American Type Culture Collection. CHLA9 cells were grown in IMDM with 20% FBS and ITS-G (Invitrogen). The FLI-BS luciferase plasmids were from Yaacov Ben-David (Chinese Academy of Sciences, Guizhou). The GRE-luciferase plasmid was from Anne Gompel (Paris Descartes University). Plasmids encoding wild type and mutant GRs were from Andrew Cato (Karlsruhe Institute of Technology, Germany). The following antibodies were used: GR (sc-393232, sc-8992), Fill (sc-356), ERG (ab133264), EWS (sc-28327), p27 (sc-36985), p16 (ab108349), γH2AX (sc-25775) and GAPDH (MAB372). Sequences of all primers are listed in Table 1. siRNA transfections used ON-Target SMART oligonucleotides from Dharmacon. Metyrapone, DEX and RU486 were purchased from Sigma-Aldrich

TABLE 1
Primers For Gluc1-ETS

| | |
|---|---|
| PU.1-Fw | TGGTGGGTCCTCCGGATTACAGGCGTGCAAAATGGAAGGG |
| | (SEQ ID NO: 1) |
| PU.1-Rev | AAACGGGCCCTCTAGATCAGTGGGGCGGGTGGC |
| | (SEQ ID NO: 66) |
| FLI1-Fw | TGGTGGGTCCTCCGGAGAC GGG ACT ATT AAG GAG GCT CTG |
| | TCG (SEQ ID NO: 2) |
| FLI1-Rev | AAACGGGCCCTCTAGACTA GTA GTA GCT GCC TAA GTG TGA |
| | AGG C (SEQ ID NO: 3) |
| ELK3-Fw | TGGTGGGTCCTCCGGAGAG AGT GCA ATC ACG CTG TGG C |
| | (SEQ ID NO: 4) |
| ELK3-Rev | AAACGGGCCCTCTAGATCA GGA TTT CTG AGA GTT TGA AGA |
| | AAG CAG TAC (SEQ ID NO: 5) |
| ERG-Fw | TGGTGGGTCCTCCGGAATT CAG ACT GTC CCG GAC CCA GC |
| | (SEQ ID NO: 6) |
| ERG-Rev | AAACGGGCCCTCTAGATTA GTA GTA AGT GCC CAG ATG AGA |
| | AGG CA (SEQ ID NO: 7) |
| ELF3-Fw | TGGTGGGTCCTCCGGAGCT GCA ACC TGT GAG ATT AGC AAC A |
| | (SEQ ID NO: 8) |
| ELF3-Rev | AAACGGGCCCTCTAGATCA GTT CCG ACT CTG GAG AAC CTC |
| | TTC C (SEQ ID NO: 9) |
| SPIC-Fw | TGGTGGGTCCTCCGGAACG TGT GTT GAA CAA GAC AAG CTG |
| | GG (SEQ ID NO: 10) |
| SPIC-Rev | AAACGGGCCCTCTAGATTA GCA ATC ATG GTG ATT TAG CTC |
| | ATG GTA ATT GG (SEQ ID NO: 11) |
| ETV2-Fw | TGGTGGGTCCTCCGGAGAC CTG TGG AAC TGG GAT GAG GC |
| | (SEQ ID NO: 12) |
| ETV-2Rev | AAACGGGCCCTCTAGATTA TTG TGT CTC TGC TCC CCG TCC G |
| | (SEQ ID NO: 13) |
| ELK4-Fw | TGGTGGGTCCTCCGGAGAC AGT GCT ATC ACC CTG TGG CAG |
| | (SEQ ID NO: 14) |
| ELK4-Rev | AAACGGGCCCTCTAGATTA TGT CTT CTG TAG GTC TGG GGA |
| | AAA TGG G (SEQ ID NO: 15) |
| GABPA-Fw | TGGTGGGTCCTCCGGAACT AAA AGA GAA GCA GAG GAG CTG |
| | ATA GAA (SEQ ID NO: 16) |
| GABPA-Rev | AAACGGGCCCTCTAGATCA ATT ATC CTT TTC CGT TTG CAG |
| | AGA AGC (SEQ ID NO: 17) |
| ELK1-Fw | TGGTGGGTCCTCCGGAGAC CCA TCT GTG ACG CTG TGG C |
| | (SEQ ID NO: 18) |
| ELK1-Rev | AAACGGGCCCTCTAGATCA TGG CTT CTG GGG CCC TGG |
| | (SEQ ID NO: 19) |
| ETV4-Fw | TGGTGGGTCCTCCGGAGAG CGG AGG ATG AAA GCC GGA TAC |
| | (SEQ ID NO: 20) |
| ETV4-Rev | AAACGGGCCCTCTAGACTA GTA AGA GTA GCC ACC CTT GGG |
| | GC (SEQ ID NO: 21) |
| ETV7-Fw | TGGTGGGTCCTCCGGACAG GAG GGA GAA TTG GCT ATT TCT |
| | CCT (SEQ ID NO: 22) |
| ETV7-Rev | AAACGGGCCCTCTAGATCA CGG AGA GAT TTC TGG CCT CTT |
| | GT (SEQ ID NO: 23) |
| ETV5-Fw | TGGTGGGTCCTCCGGAGAC GGG TTT TAT GAT CAG CAA GTC |
| | CCT (SEQ ID NO: 24) |
| ETV5-Rev | AAACGGGCCCTCTAGATTA GTA AGC AAA GCC TTC GGC ATA |
| | GGG G (SEQ ID NO: 25) |
| ETV6-Fw | TGGTGGGTCCTCCGGATCT GAG ACT CCT GCT CAG TGT AGC |
| | ATT AAG (SEQ ID NO: 26) |
| ETV6-Rev | AAACGGGCCCTCTAGATCA GCA TTC ATC TTC TTG GTA TAT |
| | TTG TTC ATC CAG (SEQ ID NO: 27) |
| ETS1-Fw | TGGTGGGTCCTCCGGAAGC TAC TTT GTG GAT TCT GCT GGG |
| | AGC (SEQ ID NO: 28) |
| ETS1-Rev | AAACGGGCCCTCTAGATCA CTC GTC GGC ATC TGG CTT GAC |
| | (SEQ ID NO: 29) |
| ETS2-Fw | TGGTGGGTCCTCCGGAGGG TCG GCT CAA TTT CAG GGC |
| | (SEQ ID NO: 30) |
| ETS2-Rev | AAACGGGCCCTCTAGATCA GTC CTC CGT GTC GGG C |
| | (SEQ ID NO: 31) |
| NFκBp65-Fw | TGGTGGGTCCTCCGGAGACGAACTGTTCCCCCTCATCTTCC |
| | (SEQ ID NO: 32) |
| NFκBp65-Fw | AAACGGGCCCTCTAGATCACCCCCTTAGGAGCTGATCTGA |
| | (SEQ ID NO: 33) |

Primers for NR-G-luc2

| | |
|---|---|
| GR-Fw | AGCACAGTGGCGGCCGCATG GAC TCC AAA GAA TCA TTA ACT |
| | CCT GGT AGA G (SEQ ID NO: 34) |
| GR-Rev | CCACCGCCACCATCGATCTT TTG ATG AAA CAG AAG TTT TTT |
| | GAT ATT TCC (SEQ ID NO: 35) |
| MR-Fw | AGCACAGTGGCGGCCGCATG GAG ACC AAA GGC TAC CAC |
| | AGT CTC C (SEQ ID NO: 36) |
| MR-Rev | CCACCGCCACCATCGATCTT CCG GTG GAA GTA GAG CGG C |
| | (SEQ ID NO: 37) |
| ERα-Fw | AGCACAGTGGCGGCCGCATGACCATGACCCTCCACACCAAAGC |
| | (SEQ ID NO: 38) |
| ERα-Rev | CCACCGCCACCATCGATGACCGTGGCAGGGAAACCCTCTGCCT |
| | CC (SEQ ID NO: 39) |
| ERβ-Fw | AGCACAGTGGCGGCCGCATGGATATAAAAAACTCACCATCTAG |
| | CCTTAATTCTCCTTCC (SEQ ID NO: 40) |
| ERβ-Fw | CCACCGCCACCATTCGATCTGAGACTGTGGGTTCTGGGAGCCCT |
| | CTTTGC (SEQ ID NO: 41) |

GR domains-G-Luc-2

| | |
|---|---|
| NTD-Fw | AGCACAGTGGCGGCCGC ATG GAC TCC AAA GAA TCA TTA |
| | ACT CCT GGT AGA GAA GAA AAC CCC (SEQ ID NO: 42) |
| NTD-Rev | CCACCGCCACCATCGAT CTT GAA TAG CCA TTA GAA AAA ACT |
| | GTT CGA CCA GGG (SEQ ID NO: 43) |
| NTD + DBD-Fw | same as NTD-Fw |
| NTD + DBD-Rev | CCACCGCCACCATCGAT CTT CCA GGT TCA TTC CAG CCT GAA |
| | GAC ATT (SEQ ID NO: 44) |
| DBD + HR + LBD- | AGCACAGTGGCGGCCGC ATG AGC CCC AGC ATG AGA CCA |
| Fw | GAT GTA AGC TCT (SEQ ID NO: 45) |
| DBD + HR + LBD- | CCACCGCCACCATCGAT CTT TTG ATG AAA CAG AAG TTT TTT |
| Rev | GAT ATT TCC ATT TGA ATA TTT TGG (SEQ ID NO: 46) |
| HR + LBD-Fw | AGCACAGTGGCGGCCGC ATG GCT CGA AAA ACA AAG AAA |
| | AAA ATA AAA GGA ATT CAG CAG GC (SEQ ID NO: 47) |
| HR + LBD-Rev | same as DBD + HR + LBD-Rev |

G-luc1-FLI1 domains

| | |
|---|---|
| NTA + 5′ETS-Fw | TGGTGGGTCCTCCGGAGACGGGACTATTAAGGAGGCTCTGTCG |
| | G (SEQ ID NO: 48) |
| NTA + 5′ETS-Rev | AAACGGGCCCTCTAGATTATTTCCCTGAGGTAACTGAGGTGTGA |
| | CAACAGC (SEQ ID NO: 49) |
| NTA + 5′ETS + FLS- | same as NTA + 5′ETS-Fw |
| Fw | |
| NTA + 5′ETS + FLS- | AAACGGGCCCTCTAGATTAGGAGAGCAGCTCCAGGAGGAATTG |
| Rev | CCACAG (SEQ ID NO: 50) |

| | |
|---|---|
| NTA + 5′ETS + FLS + | same as NTA + 5′ETS-Fw |
| 3′ETS-Fw | |
| NTA + 5′ETS + FLS + | AAACGGGCCCTCTAGATTACTGCTGGTGGGCATGGTAGGA |
| 3′ETS-Rev | (SEQ ID NO: 51) |

PCR validation

| | |
|---|---|
| PDIA6-Fw | GGACACTGCAAAAACCTAGAGC (SEQ ID NO: 52) |
| PDIA6-Rev | CCAGAACCTGATTGACTGTAGCA (SEQ ID NO: 53) |
| COL6A3-Fw | CATAACCGCTGTGCGGAAAAT (SEQ ID NO: 54) |
| COL6A3-Rev | TCATCTAGGGACTTACCACCTG (SEQ ID NO: 55) |
| TMED10-Fw | GCCCTTTTCCGTTAGCGTTC (SEQ ID NO: 56) |
| TMED10-Rev | ACTTGCGAGAGTTATTGGGCA (SEQ ID NO: 57) |
| SEC61G-Fw | GCAGTTTGTTGAGCCAAGTCG (SEQ ID NO: 58) |
| SEC61G-Rev | CCAGCCGAATGGAGTCCTT (SEQ ID NO: 59) |
| PPA1-Fw | CCCTGGAGTACCGAGTCTTCC (SEQ ID NO: 60) |
| PPA1-Rev | CATTTTTGCATTAGACCAGCGTG (SEQ ID NO: 61) |
| IGFBP7-Fw | CGAGCAAGGTCCTTCCATAGT (SEQ ID NO: 62) |
| IGFBP7-Rev | GGTGTCGGGATTCCGATGAC (SEQ ID NO: 63) |
| RPL37A-Fw | CCAAACGTACCAAGAAAGTCGG (SEQ ID NO: 64) |
| RPL37A-Rev | GCGTGCTGGCTGATTTCAA (SEQ ID NO: 65) |

Protein-fragment complementation assays (PCA): PCA was performed as previously described [18]. Briefly, HEK-293 cells were reverse transfected in white 96-well tissue culture plates with Gluc-1 and Gluc-2 plasmids (25 ng, each) using the JetPEI reagent. Cells were starved and treated with DEX (1 μM) prior to extraction.

Luciferase-reporter assays: Cells were co-transfected with a luciferase plasmid containing the consensus glucocorticoid response element (GRE) along with pGL3-Control (Promega, Madison, Wis.). Luciferase activity was determined using the dual-luciferase reporter assay system (Promega). Firefly luciferase luminescence values were normalized to Renilla luminescence.

Proximity ligation assay (PLA), extraction of cells, co-immunoprecipitation and cell migration/invasion assays: These assays were performed exactly as recently reported [53].

ES animal model studies: All animal experiments were approved by the Weizmann Institute's Animal Care and Use Committee. RD-ES cells and A673 cells (2.5×10⁶) were inoculated subcutaneously in the right legs of female SCID mice (6 weeks old). Tumor growth was monitored once every 3 days using a caliper. Once tumor volume reached approximately 150 mm³, mice were intraperitoneally treated once per day with DEX or RU486 (both at 1 mg/kg), or metyrapone (25 mg/kg). Mice were euthanized when tumor size reached 800-900 mm³. For metastasis assays, luciferase-labeled TC-71 cells (10⁶) were injected orthotopically into the tibia of female SCID mice. Once the primary tumor reached the allowed size limit, we excised the lungs and examined metastases using luciferase bioluminescence imaging.

RNA isolation and real-time PCR analysis: Total RNA was extracted using the PerfectPure RNA Cultured Cell Kit (5-prime, Hamburg) and RNA quantity and quality were determined using the NanoDrop ND-1000 spectrophotometer (Thermo Fischer Scientific, Waltham, Mass.). Complementary DNA was synthesized using the High Capacity Reverse Transcription kit (Applied Biosystems, Carlsbad, Calif.). Real-time qPCR analysis was performed using SYBR Green (Applied Biosystems) and specific primers on the StepOne Plus Real-Time PCR system (Applied Biosystems). qPCR signals (cT) were normalized to beta2-microglobulin (B2M).

Immunofluorescence analysis of tumor sections and cultured cells: Formalin-fixed and paraffin-embedded (FFPE) tumor sections (width: 2 μm) were de-paraffinized in xylene and rehydrated in graded ethanol. Antigen retrieval was performed using a citric acid solution (pH 9.0) in a microwave. After washing, slides were blocked in saline containing 20% horse serum, followed by treatment for 15 minutes with an avidin/biotin blocking solution, and an overnight incubation with the corresponding primary antibody. Slides were incubated for 12 hours at room temperature followed by incubation for 24 hours at 4° C. Thereafter, sections were incubated for 90 minutes with a biotinylated secondary antibody, followed by a Cy3-conjugated StreptAvidin. Next, tumor sections were washed and nuclei were stained with DAPI. Finally, each slide was covered by a coverslip with Aqua Polymount and examined on the next day using a fluorescence microscope (Eclipse Ni-U, Nikon, Tokyo, Japan) equipped with a Plan Fluor objectives (6×), connected to a monochrome camera (DS-Qi1, Nikon). KI67 positive cells were counted using the Image Pro Plus software. To determine localization of GR and FLI1 in cultured cells, the medium of DEX-treated were washed in saline containing Tween 20 (0.1%; w/v; PBS-T) and fixed overnight at 4° C. in formaldehyde (4%). Next, cells were washed and permeabilized for 7 minutes (saline with 0.1% TritonX-100). Next, blocking was carried out for 30 minutes using 2% fetal bovine serum, followed by an overnight incubation at 4° C. with a primary antibody in PBS-T containing FBS (1%). This was followed by FITC-conjugated secondary antibody and DAPI for 45 minutes in dark. Images were captured using a Zeiss 710 confocal microscope (40× magnification) and processed using the Zeiss ZEN2011 software.

Nuclear and cytoplasmic fractionation: Cell pellets were lysed in 0.1 ml cytoplasmic lysis buffer (10 mM HEPES pH 7.9, 10 mM KCl, 0.1 mM EGTA, 0.1 mM EDTA, 1 mM DTT, 0.5% NP-40, with protease and phosphatise inhibitors). The cytoplasmic fraction was collected by centrifugation (600 g for 5 minutes). Nuclei were washed and resuspended in 50 μl of nuclear lysis buffer (20 mM HEPES pH 7.9, 0.4 M NaCl, 1 mM EGTA, 1 mM EDTA, 1 mM DTT, protease and phosphatase inhibitors) by repeated freezing and thawing. Supernatants containing the nuclear fraction were collected by centrifugation at 12,000 rpm for 20 minutes.

Apoptosis assays: Assays were performed using the FITC Annexin V Apoptosis Detection Kit with 7-AAD (from BioLegend) and analyzed using a BD FACSAria Fusion instrument controlled by BD FACS Diva software v8.0.1 (BD Biosciences).

Colony formation and adhesion assays: ES cells (150-300) were seeded in 6-well plates. Ten days after treatment, cells were washed, fixed in paraformaldehyde (4%) and then stained for 60 minutes with crystal violet. Cells were then photographed using a binocular microscope and analyzed using ImageJ (NIH, USA). For adhesion tests, plates were coated overnight with Cultrex™ RGF BME (R&D Systems) and gently washed thereafter (0.1% albumin in medium). RD-ES and TC-71 cells (30,000 cells/well) were allowed to adhere to the substrate for 8 hours at 37° C. CHLA9 cells were seeded in non-coated plates and allowed to attach for 90 minutes. Unattached cells were removed and adherent cells were rinsed, fixed with paraformaldehyde (4%), and quantified using crystal violet staining (0.1%). The optical density was measured at 550 nm.

Identification of a prognostic gene signature: A list of 3,709 genes that mapped to GR peaks was derived from a previously reported GR CHIP-Seq analysis (GSE65847). Three datasets that analyzed gene expression in Ewing's sarcoma (GSE12102, n=37, GSE34620, n=117 and GSE17618, n=44), along with an osteosarcoma dataset (GSE14827, n=27), enabled selection of 58 GR-regulated genes from the 3709 gene list that showed the highest variation in expression compared to the mean expression levels of all genes in the datasets examined. Patient survival data from two datasets, GSE34620 (117 patients) and GSE17618 (44 patients) were used as discovery and validations sets, respectively. The statistical effect of gene expression on the survival of the 117 sample set (48 events; GSE34620) were analyzed using the My.stepwise.coxph function from the “My.stepwise” R package. This allowed us to select the most informative gene expression variables and derive the following formula for the metagene: Metagene=[1.2176*Expr(PDIA6)−0.6061*Expr (COL6A3)−1.2175*Expr(TMED10)+1.6618*Expr(SEC61G)+0.5134*Expr(PPA1)+0.3943*Expr(IGFBP7)+1.9170*Expr(RPL37A)]/sum(coeff.metagene)

With coeff.metagene=(1.2176, −0.6061, −1.2175, 1.6618, 0.5134, 0.3943, 1.9170.

Note that expression threshold levels were selected in a way that maximized the difference between the two survival curves. Statistical testing in the framework of the semi-parametric cox proportional hazards model using only the metagene variable and the coxph function from the R package “survival yielded a P-value of 1.54e-12. For validation, we performed the same patient survival analysis and used the same metagene, but referred to the GSE17618 cohort (44 patients and 26 events) and applied optimization of expression thresholds (with minimal sample size=total_cohort_size/4). The semi-parametric cox proportional hazards model using only the metagene variable yielded a P-value of 0.000114.

Statistical analysis: All data were analyzed using the Prism Graphpad software and R. Statistical analyses were performed using t-test and one- or two-way ANOVA with Tukey's or Dunnett's test (*p≤0.05; **p≤0.01; ***p≤0.001).

Results

Protein-Fragment Complementation Assays (PCA) Reveal Physical Interactions Between GR and Specific Members of the ETS Family

Because trans-repression by GR involves complex formation with major TFs, such as NF-κB, AP-1 and NGF1-B, it was hypothesized that ETS family TFs are similarly controlled by GR. To test this prediction, a protein-fragment complementation assay was used (PCA). PCA employs two structurally inactive fragments of a reporter luciferase, which are separately fused to two proteins of interest (e.g., GR and a member of the ETS family). Essentially, physical interactions between the two TFs might enable the reporter to regain activity. The present assays used a previously described adaptation of the Gaussia luciferase (Gluc) reporter assay [18]. To enable PCA, Gluc was split into an amino-terminal fragment, Gluc1 (amino acids 1-93) and a carboxyl-terminal fragment, Gluc2 (amino acids 94-169; FIG. 1A). A library comprising sixteen ETS factors, all fused in frame downstream to Gluc1, was constructed. Likewise, Gluc2 was fused in frame to the carboxyl terminus of GR. As control, Gluc2 was also fused to the tails of three other NRs: estrogen receptor alpha (ERα), estrogen receptor beta (ERβ) and mineralocorticoid receptor (MR; FIG. 1A and Table 2, herein below).

TABLE 2
ETS-NR PCA Library

| | | | Fusion to luciferase | | |
|---|---|---|---|---|---|
| | | | fragment | | |
| | | | X- | X- | GLuc1- |
| No. | Gene | Accession Number | GLuc1 | GLuc2 | X |
| 1 | GR | NM_000176.2 | + | + | |
| 2 | MR | NM_000901.4 | + | + | |
| 3 | ERβ | NM_001437.2 | + | + | |
| 4 | ERα | NM_000125.3 | + | + | |
| 1 | PU.1 | NM_001080547.1 | | | + |
| 2 | FiI1 | NM_002017.4 | | | + |
| 3 | ELK3 | NM_005230.2 | | | + |
| 4 | ERG | NM_001136154.1 | | | + |
| 5 | ELF3 | NM_001114309.1 | | | + |
| 6 | SPIC | NM_152323.1 | | | + |
| 7 | ETV2 | NM_014209.3 | | | + |
| 8 | ELK4 | NM_001973.3 | | | + |
| 9 | GABPA | NM_002040.3 | | | + |
| 10 | ELK1 | NM_001114123.2 | | | + |
| 11 | ETV4 | NM_001079675.2 | | | + |
| 12 | ETV7 | NM_016135.3 | | | + |
| 13 | ETV5 | NM_004454.2 | | | + |
| 14 | ETV6 | NM_001987.4 | | | + |
| 15 | ETS1 | NM_001143820.1 | | | + |
| 16 | ETS2 | NM_001256295.1 | | | + |

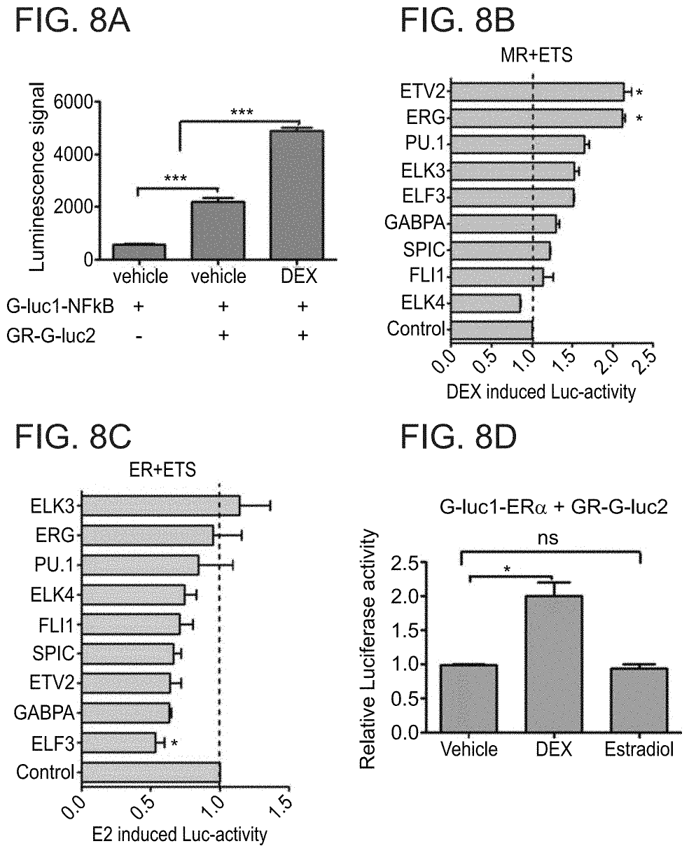

The library of Gluc1-ETS plasmids was transfected into HEK-293 cells, along with the Gluc2-GR plasmid. Following 24 hours of incubation, cells were starved for serum factors, and thereafter they were treated for 60 minutes with the vehicle solvent or with dexamethasone (DEX; 1.0 μM). The resulting luminescence was normalized to the signal associated with cells treated with vehicle only. As summarized in FIG. 1B, ERG, as well as its closest homologue, FLI1, and the more distantly related PU.1 and ETV4, showed highly significant increases in luciferase activity when cells were challenged with DEX. To corroborate these observations, several tests were used. Firstly, the present inventors confirmed that their protocol could detect interactions between Gluc1-NF-κB and GR-Gluc2 (FIG. 8A). Secondly, to test inducibility, cells were treated with either DEX or a combination of DEX and RU486, a GR antagonist. As shown in FIG. 1C, RU486 significantly inhibited DEX-induced physical interactions between GR and FLI1, PU.1 and ERG, but only a trend was noted when the weaker binder, ETV4, was tested. To confirm the physical interactions in naïve cells, a co-immunoprecipitation assay was carried out (FIGS. 1D and 1E). The results indicated that the endogenous form of GR physically binds with both FLI1 and ERG in living cells. Unlike the interaction with ERG, which was enhanced following treatment with DEX, the GR-FLI1 interaction existed prior to treatment with DEX. Nevertheless, treatment with RU486 strongly reduced co-immunoprecipitation of GR and the two ETS proteins that were tested.

The adrenal corticosteroids (i.e., glucocorticoids and mineralocorticoids) play important but largely distinct physiological roles [19]. Thus, MR is highly sensitive to both mineralocorticoids and glucocorticoids, while GR responds to glucocorticoids but displays insensitivity to mineralocorticoids. To address potential interactions between MR and ETS factors, the ability of DEX to activate MR was utilized. Similarly, a PCA was performed with Gluc2 proteins fused to ERα and the effects of estradiol (E2, an ER ligand) were examined. The results established differential specificity and strength of interactions between nuclear receptors and various ETS family members (Supplementary FIGS. 8B-C). For example, DEX stimulated strong interactions between MR and both ETV2 and ERG, which differed from the pattern displayed by GR, but no ETS factor showed interactions with ERα. On the contrary, treatment with E2 significantly inhibited the interaction between ERα and ELF3 (FIG. 8C), an ETS family member that acts as a transcriptional repressor of ERα. In line with this, it has recently been reported that ELF3 binds with ERα in the absence of E2, but ELF3 dissociation occurs upon E2 treatment [20]. Further studies using co-transfected GR-Gluc2 and ERα-Gluc1 detected a significant increase in DEX-induced luciferase activity, but treatment with E2 exerted no marked effect (FIG. 8D). This observation indicates that GR undergoes a ligand-induced alteration that promotes GR-ERα interactions, but a similar process may not occur in response to E2. Consistent with this interpretation, it has been reported that endogenous GR interacts with ERα in mammary cells [21]. In summary, the tests that were performed established specificity, sensitivity and reliability of the PCA, as well as the interactions between certain ETS factors and the active form of GR.

FLI1 and Active GRs Form a Physical Complex in Living Cells and Translocate to the Nucleus

Next, the kinetics and localization of FLI1-GR interactions were explored. PCA performed at increasing time intervals following DEX treatment demonstrated that GR-FLI1 interactions peaked 60 minutes after DEX stimulation, but decayed thereafter (FIG. 2A). Likewise, ERG signals peaked earlier, but in similarity to FLI1, the signal started decaying 30 minutes later. Presumably, delayed interactions between GR and ETS factors is due to sub-cellular compartmentalization. To test this, GR and FLI1 were immunostained in HEK-293 cells following DEX treatment (FIG. 2B) and subcellular fractionation using markers of the nuclear (histone 3) and cytoplasmic fractions (GAPDH; FIG. 2C) was also performed. Evidently, GR translocated to the nucleus within 10 minutes but at 30 minutes some nuclei were already emptied. In parallel, FLI1 was basally cytoplasmic, but 30 minutes after stimulation it started occupying nuclei. In similarity to GR, FLI1 eventually localized to the cytoplasm. These observations corroborate the kinetics of protein-protein interactions presented in FIG. 2A, and propose that they are not only inducible and transient, but are confined to nuclei and take place 30-60 minutes after DEX stimulation.

Dexamethasone Enhances Interactions Between the DBD-HR Domain of GR and the FLS Domain of FLI1

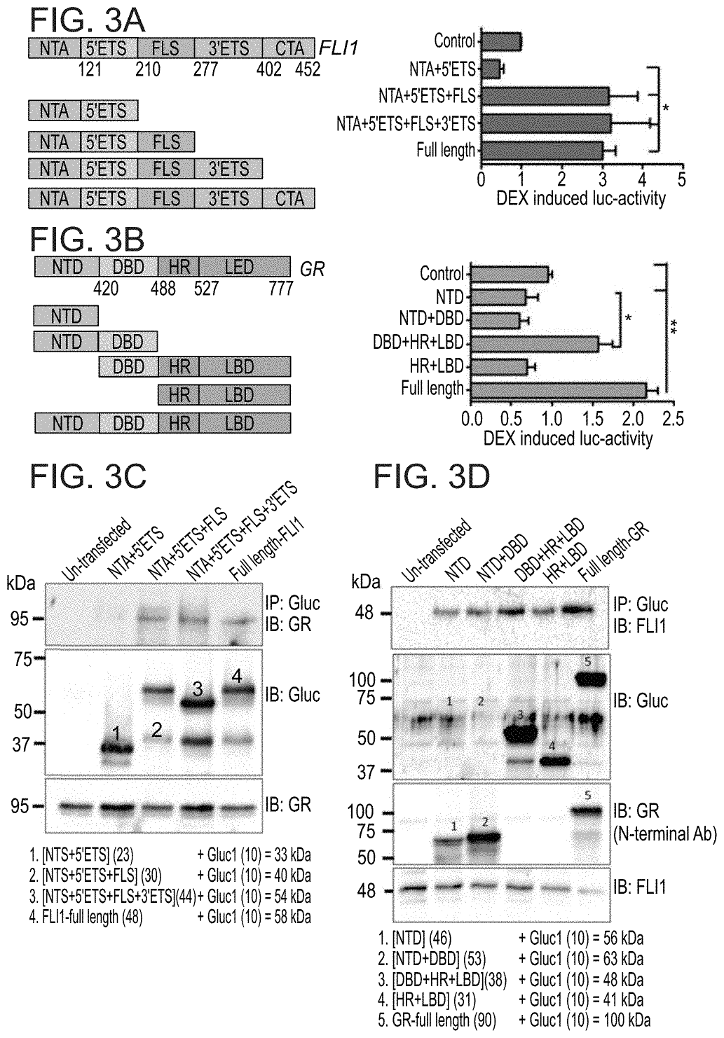

To map domains of FLI1 and GR that are engaged upon DEX stimulation, deletion mutants of both TFs (FIGS. 3A and 3B) were constructed. Gluc1-FLI1 proteins encompassing two, three, four or all five FLI1's domains were transfected together with a plasmid encoding Gluc2-GR. The PCA signals attributed recognition to the FLI1-specific (FLS) region. Similar analyses that dissected the four domains of GR mapped FLI1 recognition to the DNA binding domain (DBD) and the flanking hinge region (HR). Congruently, it has been reported that the p65 subunit of NF-κB physically interacts with the DBD of GR [22]. To validate FLS binding to DBD/HR, co-immunoprecipitation assays in HEK-293 cells were performed. The results confirmed the conclusions (FIGS. 3C and 3D): Gluc1-FLI1 proteins containing the FLS region retained binding with endogenous GR, but NTA+5′ETS, a mutant lacking the FLS, lost this attribute. Similarly, a GR-Gluc2 protein comprising DBD, HR and LBD co-immunoprecipitated FLI1, but the NTD, neither alone nor in combination with the DBD, recognized FLI1. In conclusion, the recognition between GR and FLI1 maps to discrete domains: the unique FLI1-specific region (FLS) and the centrally located DBD-HR of GR.

Inducible Interactions Between FLI1 and DNA-Bound GR Augment Transcriptional Activity of the Steroid Receptor

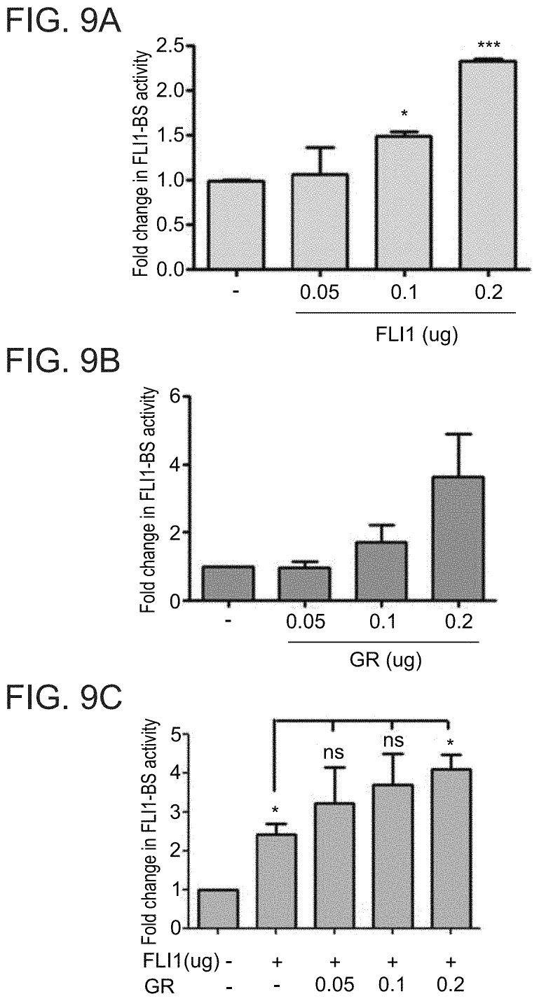

While the classical mode of GR transactivation involves recognition of the GRE and transcription from proximal promoters, an alternative mode permits GR to localize to additional genomic sites through binding with other TFs [25] Likewise, the FLI1's target sequence GGA(A/T) is shared by all ETS factors [23], but tethering modes are less characterized. To examine the possibility that the uncovered GR-FLI1 interactions regulate transcription via the tethering model, two luciferase reporter systems were performed: (i) the FLI1-binding sequence (BS) and, (ii) a GRE-driven reporter. In line with the classical model, upon co-transfection of each reporter and a plasmid encoding the respective TF, dose-dependent increases in luciferase activity were observed (FIG. 4A and FIG. 9A). Consistent with other tethering models, increasing the amount of the FLI1-encoding plasmid induced moderate but reproducible and dose-dependent enhancement of expression from the GRE (FIG. 4B), but a reciprocal experiment, which used FLI1-BS and a GR plasmid, achieved no statistical significance (FIG. 9B).

Notably, co-expression of GR and FLI1 caused only a small increase in transcription from FLI1-BS (FIG. 9C). In contrast, a relatively small amount of ectopic FLI1 strongly enhanced GRE activity, beyond the effect of GR alone (FIG. 4C). To validate tethering of FLI1 to DNA-bound GR, a GR mutant, GRdim⁻ (A458T) [26] was used. Mutagenesis of alanine 458, which resides in the DBD, decreases GRE-mediated transactivation by diminishing dimerization and DNA binding (Jewell et al, 2012). As expected, wild-type GR showed a large (17-fold) increase in GRE activity, but ectopic expression of GRdim⁻ showed a much weaker effect on GRE activity (FIG. 4D). This observation indicates that GR's DNA binding is important for the ability of FLI1 to trans-activate transcription from the GRE. Next, endogenous GR was addressed (FIG. 4E): GRE activity increased by 40-fold following treatment of naïve cells with DEX, but co-treatment with RU486 was inhibitory. Furthermore, the luciferase signal almost doubled when a FLI1 plasmid (0.1 μg) was introduced into cells. In conclusion, the present results are consistent with FLI1-mediated cross-activation of GR's target genes. Conceivably, the underlying mechanism entails physical recruitment of FLI1 to DNA-bound dimers of GR, and results in enhanced transcription from GRE sites (see FIG. 4F).

GR Forms a Complex with the Oncogenic EWS-FLI1 Fusion Protein in Ewing's Sarcoma (ES) Cells

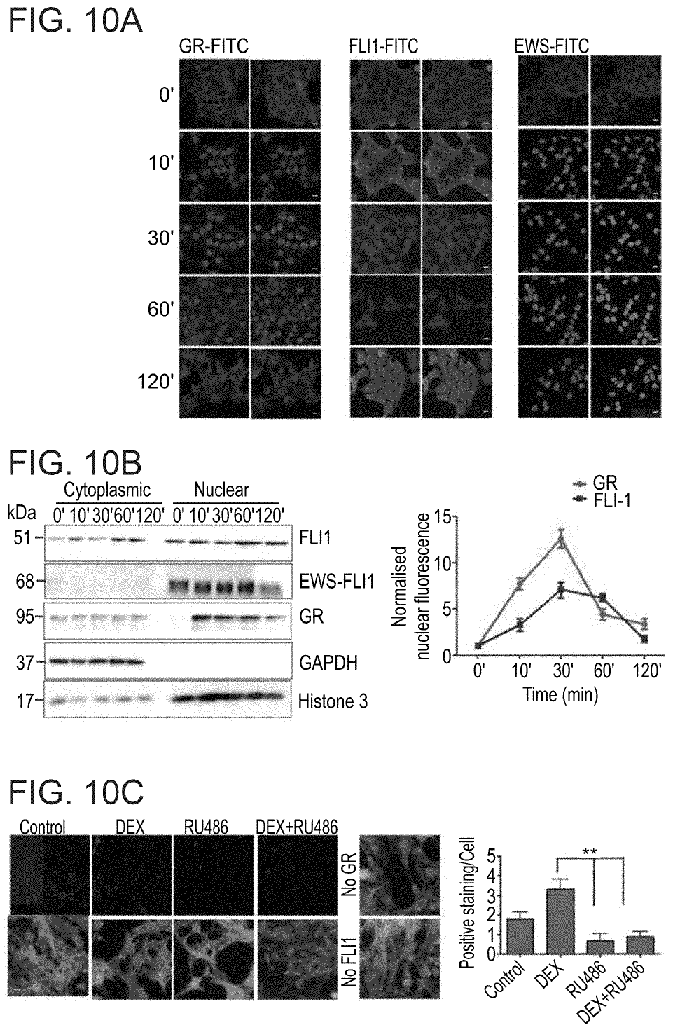

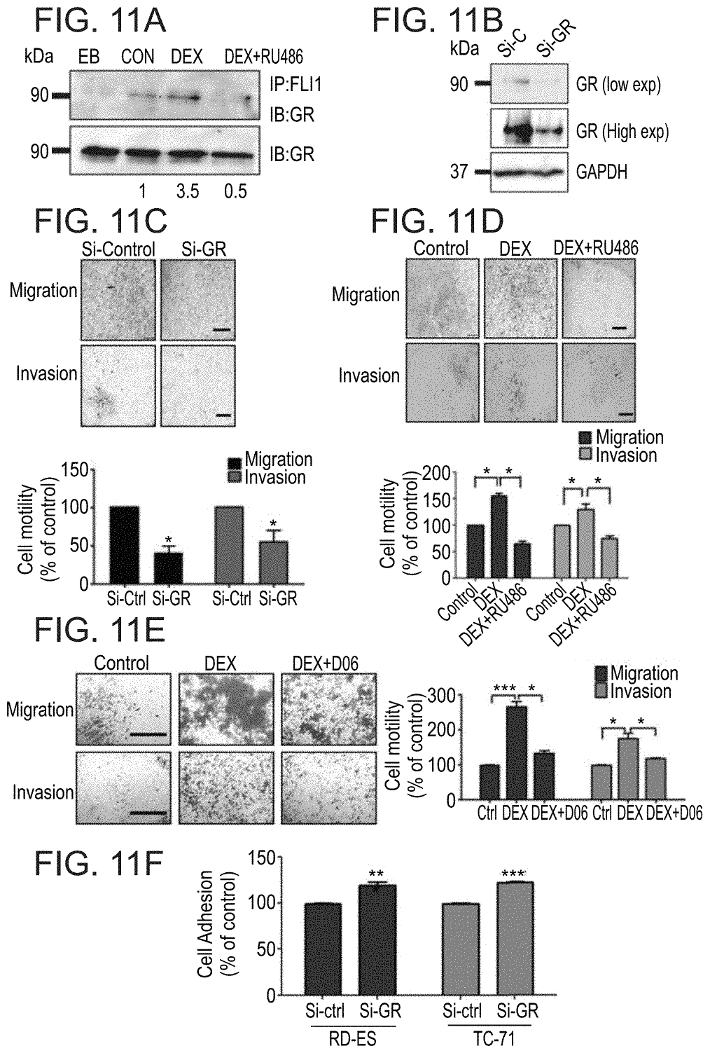

Genes encoding ETS family TFs, such as FLI1 and ERG, are frequently deregulated in cancer [27]. Approximately 50% of all prostate cancers harbor hybrid ERG genes [28, 29], and replacement of the N-terminus of FLI1 by the transactivation domain of the RNA-binding EWS protein characterizes most Ewing's sarcomas (ES) [16]. Because the resulting EWS-FLI1 fusion retains the GR binding site, it was hypothesized that GR and EWS-FLI1 collaborate in ES. Firstly, the present inventors demonstrated time- and DEX-dependent translocation of GR to nuclei of A673 ES cells, which constitutively harbor EWS-FLI1 (FIGS. 10A-B). Next, physical complex formation between GR and EWS-FLI1 was verified by applying the proximity ligation assay (PLA). This test employs antibodies specific to proteins of interest, as well as secondary antibodies attached to oligonucleotides, which form circular DNA strands when bound in close proximity [30]. Importantly, strong protein-protein interactions were observed, which were significantly enhanced by DEX and blocked by RU486 (FIG. 10C). Congruent with complex formation, co-immunoprecipitation assays validated protein-protein interactions between GR and EWS-FLI1. This test examined the endogenous GR of two ES cell lines, CHLA9 (FIG. 5A) and RD-ES (FIG. 11A). Note that prior to extraction of cells they were treated with DEX or DEX plus RU486. As shown, an antibody specific to EWS-FLI1 was able to detect the fusion protein in GR immunoprecipitates. Although pre-treatment with DEX did not change the amount of pulled-down hybrid protein, incubations with RU486 completely (CHLA9 cells) or partially (RD-ES cells) inhibited the interactions, as predicted by the physical complex model.

Activation of GR Increases Migration and Invasion of ES Cells

Cell migration may reflect the interactions between GR and EWS-FLI1. To test this, GR's abundance was reduced by using siRNAs (FIG. 5B and Supplementary FIG. S4B) and observed reduced migration and matrix invasion (FIGS. 5C and 11C). Reciprocally, pre-treatment with DEX enhanced these function of EWS-FLI1-driven cells (FIGS. 5D and 11D). Remarkably, treatment with RU486 and D06, a nonsteroidal antagonist, inhibited migration and invasion (FIG. 11E). As a complementary approach, EWS-FLI1 was depleted in CHLA9 cells (FIG. 5E). The cells were later stimulated with DEX. Congruent with the proposed GR and EWS-FLI1 collaboration, the specific siRNA reduced both basal and DEX-induced cell migration (FIG. 5F). Notably, genes encoding focal adhesion and extracellular matrix regulatory proteins are dominant targets of EWS/FLI-mediated transcriptional repression [34]. In line with these observations, GR depletion increased adhesion of CHLA9 cells to fibronectin (FIG. 5G), as well as adhesion of both RD-ES and TC-71 cells to basement membranes rich in laminin and collagen (FIG. 11F).

Another set of experiments made use of CHLA9 cells and vectors encoding GR or the dimerization-defective mutant, GRdim⁻. Following transfection, cells were plated on matrix-coated or uncoated filters. Overexpression of GR increased migration and invasion of CHLA9 cells, but the mutant was less effective (FIG. 5H). In conclusion, the present data proposes the following scenario: EWS-FLI1 physically binds with active GRs and enhances transcription of glucocorticoid-inducible genes able to reduce matrix adhesion, while promoting ES cell migration and invasion.

Activation of GR Increases Proliferation and Inhibits Apoptosis of ES Cells

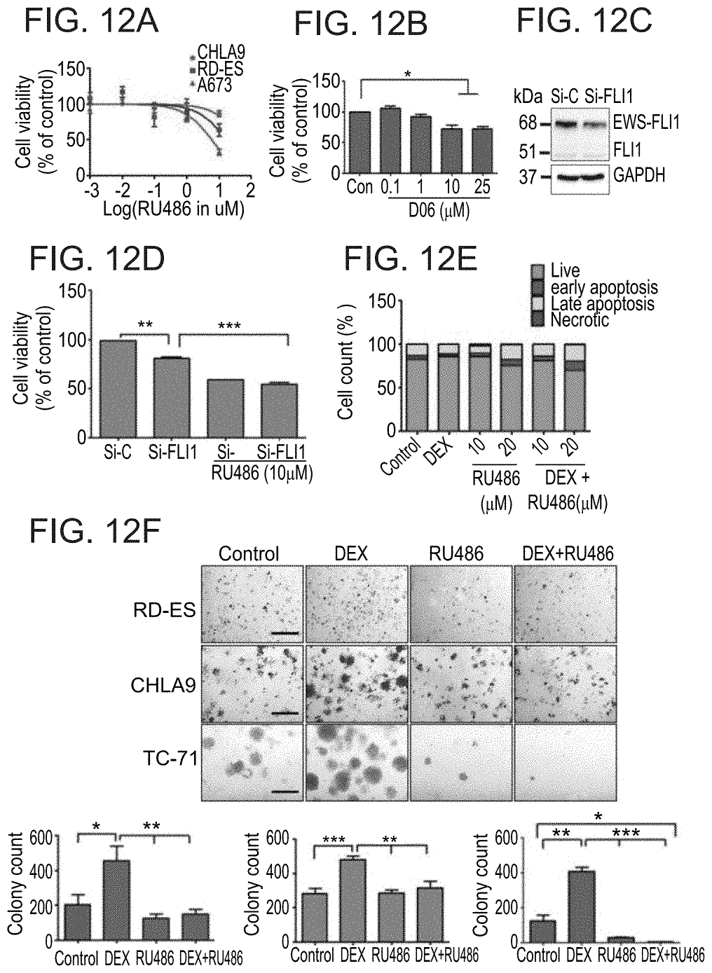

According to recent reports, the EWS-FLI1 fusion promotes cell cycle progression and inhibits apoptosis [31, 35]. To test involvement of GR, the present inventors assayed the conversion, by viable cells, of MTT (3-(4,5-dimethylthiazol-2-yl)-2,5-diphenyltetrazolium bromide) to an insoluble formazan. In line with the proposed collaboration between EWS-FLI1 and GR, following treatment with RU486 or a non-steroidal (D06) GR antagonist a dose-dependent reduction in survival of three ES cell lines was observed (FIGS. 12A-B). Next, siRNA specific to the FLI1 region of the fusion (FIG. 12C) were analyzed in A673 cells. This second MTT assay demonstrated that EWS-FLI1 knockdown significantly reduced ES cell survival and RU486 further reduced the fraction of metabolically active cells FIG. 12D). To help distinguish between early and late apoptosis, 7-amino-actinomycin D (7-AAD) was used. Interestingly, while treatment with DEX almost eliminated the fraction of cells undergoing early apoptosis, RU486 increased both early and late apoptosis (FIG. 12E), indicating that GR promotes ES cell growth and survival. This was supported by colony formation assays performed with three ES cell lines (FIG. 12F). In summary, the activated form of GR clearly collaborates with the oncogene of ES, EWS-FLI1, in terms of supporting cell proliferation, overcoming apoptosis and inducing various aspects of cell motility.

Cortisol Lowering and Treatments with an Antagonist of GR Inhibit Growth of Ewing's Sarcoma Xenografts

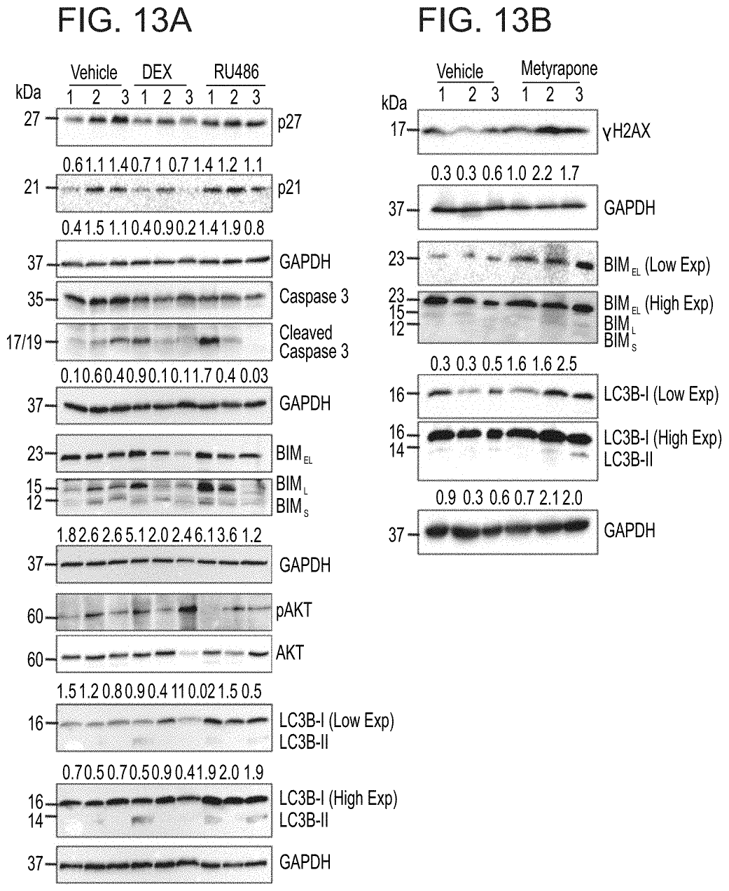

Because inhibition of GR retarded survival of ES cells, the present inventors asked whether DEX and RU486, clinically approved GR agonist and antagonist, respectively, can modify the tumorigenic growth of an ES xenograft. Once RD-ES tumors that were pre-implanted in mice became palpable, animals were randomized in three groups, which were treated with vehicle, DEX or RU486. Unlike mice treated with DEX or vehicle, significantly slower tumor growth was observed in animals treated with RU486 (FIG. 6A). In line with GR-supported tumorigenesis, staining of tumor sections with an antibody to KI67, a proliferation marker, indicted slower proliferation rates in RU486-treated tumors, and stronger KI67 positivity of DEX-treated tumors relative to the control group (FIG. 6B). Immunoblotting of extracts prepared from three tumors of each group are shown in FIG. 13A. Despite inter-animal variation, two inhibitors of the cell cycle, p21 and p27, displayed increased abundance in RU486-treated tumors. RU486-treated tumors also displayed up-regulation of the larger isoform of BIM, an initiator of apoptosis. Notably, under normal conditions the large isoforms, especially BIM_{EL}, are sequestered by microtubules, but once freed they negate the anti-apoptotic activity of BCL-2 in a caspase-independent manner [36]. While no clear activation of caspase 3 was detected, the data implied that the upstream AKT survival pathway was inhibited by the GR antagonist. Because BIM serves as a molecular link between apoptosis and autophagy [37], LC3, an autophagy marker was also probed. An increased abundance of LC3B-I in RU486-treated tumors was observed. Taken together, these observations suggest that RU486 inhibits ES tumors by means of a caspase 3 independent pathway that harnesses both apoptosis and autophagy.

Assuming that murine cortisol in the tumor microenvironment activates GR in ES xenograft models, the effect of cortisol-lowering was tested on the tumorigenic growth of another ES model, A673 (FIGS. 6C and 6D). Cells were implanted in SCID mice and, when tumors became palpable, mice were randomized into a control group and a cohort (n=9) that was treated with metyrapone, a clinically approved cortisol-lowering inhibitor of the steroidogenesis enzyme 11β-hydroxylase. A clear and statistically significant decrease of tumor growth rates was associated with the lowered levels of cortisol. Analyses of tumor sections revealed metyrapone-induced effects on BIM_{EL} and LC3B-I, in similarity to the effects observed with RU486, along with elevated γH2AX, a component of the DNA damage response (FIG. 13B) [38]. Taken together, by using two xenograft systems and two different GR-inactivating strategies, the present results support a model attributing to GR the ability to enhance tumorigenicity of sarcomas driven by EWS-FLI1

Inactivation of GR Impedes Bone-to-Lung Metastasis in ES Animal Models

Unlike ES patients with localized disease at diagnosis, who have 5-year survival rates approaching 70%, patients with metastatic disease have dismal outcomes with 5-year survival rates of 15-25% [39]. Because the present in vitro tests attributed important roles in cell motility to the GR-FLI1 interaction, and a previous report demonstrated that detachment from bone is an essential EWS-FLI1 regulated step towards metastasis [40], the present inventors examined metastasis in animal models. TC-71 cells pre-engineered to express luciferase were injected into the tibia of SCID mice, which were treated with either DEX or RU486. FIG. 6E shows whole lungs from representative animals from each group, along with luminescence signals from all mice. Despite inter-animal variation, this analysis indicated that GR activation associated with enhanced bone-to-lung metastasis, whereas blocking GR correlated with lessened lung nodules.

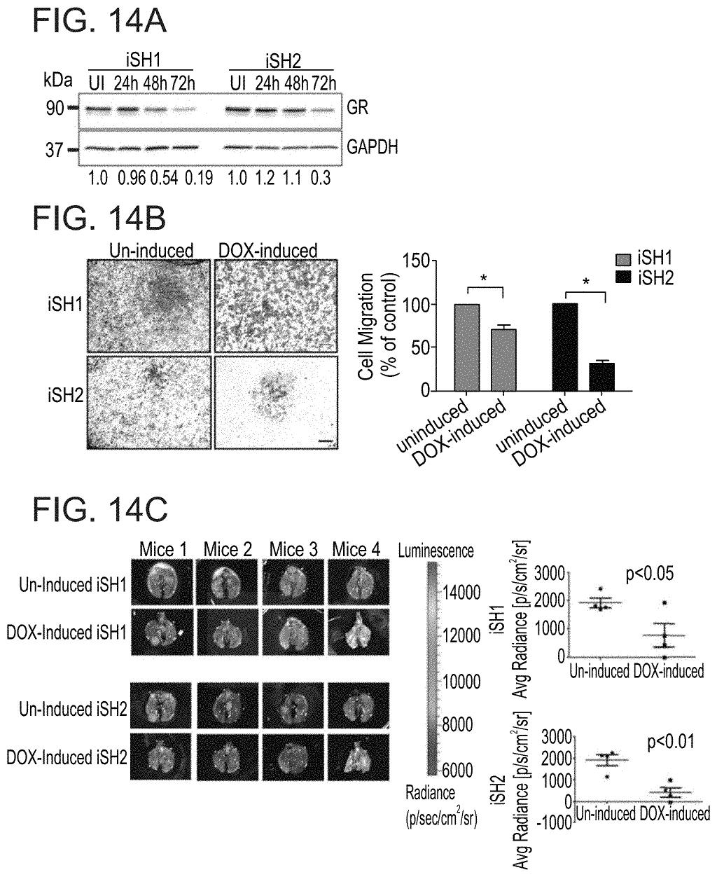

To further relate metastasis to GR, the present inventors employed inducible lentiviral shRNAs specific to GR. Two different shRNAs were used to transduce TC-71 cells, inducible sh1 (iSH1) and iSH2. When the corresponding sub-lines of TC-71 were grown in medium supplemented with the inducer, doxycycline (DOX), time-dependent decreases in GR expression levels (FIG. 14A) and lower capacity to migrate (FIG. 14B) were observed. Next, both sub-clones were implanted in the tibia of SCID mice. Once tumors reached a certain volume, mice were randomly divided into two groups, which were orally treated with either saline (un-induced group) or DOX (induced group). When the primary bone tumor reached 10% of body weight, lungs were excised and analyzed for metastasis using bioluminescence imaging. The results presented in FIG. 14C indicated that the GR-depleted clones partially lost their ability to dissociate from bones and colonize lungs. Hence, it can be concluded that GR activity is essential for dissemination of metastases, in line with a model attributing to the oncogene of ES, EWS-FLI1, the ability to collaborate with GR.

Identification of a GR Target Gene Signature Able to Predict Survival of ES Patients

The inferred ability of EWS-FLI1 to augment GR's transcriptional activity, in favor of invasive growth, prompted the present inventors to identify a prognostic, GR-regulated gene signature. Because the genomic binding sites of GR in ES are still unknown, the present inventors referred to analyses of chromatin immunoprecipitation of GR coupled to massively parallel DNA sequencing (ChIP-Seq) previously performed with osteosarcoma cells (GSE65847). This identified 3,709 genes that mapped to GR peaks. Subsequently, an osteosarcoma patient dataset (GSE14827, n=27) was used, along with three ES clinical datasets of gene expression (GSE12102, n=37, GSE34620, n=117 and GSE17618, n=44). These four sets enabled the selection of 58 GR-regulated genes (Table 3), which showed the most extreme changes in expression compared to the mean expression levels of the genes in all datasets examined.

TABLE 3

| Gene name | Gene name |
|---|---|
| A2M | PRDX6 |
| ACTB | MAFB |
| ANXA1 | PDIA6 |
| ANXA2 | ATP6AP2 |
| ANXA5 | BASP1 |
| CALM2 | PNRC1 |
| COL1A2 | TMED10 |
| COL6A3 | USP22 |
| DAD1 | ISCU |
| DPYSL2 | SEC61G |
| IARS | MMADHC |
| IGFBP7 | HSD17B12 |
| MT2A | NDUFA12 |
| PPA1 | GOLPH3 |
| PSMA6 | TUBB6 |
| PSMB4 | MRFAP1 |
| RPL3 | ZNF664 |
| RPL37A | IGLL1 |
| S100A10 | PDE6C |
| SELENOP | PF4V1 |
| SRP9 | SLC13A1 |
| UBE2E1 | USH2A |
| YWHAZ | MCF2L2 |
| ZFAND5 | MORC1 |
| EIF3D | PCDH15 |
| EIF3H | ABCA13 |
| COX7A2L | KIAA0825 |
| TMSB10 | EYS |
| MRPL33 | IYD |

Patient survival data from two datasets, GSE34620 (117 patients) and GSE17618 (44 patients), were used as training and validation sets, respectively. Gene-by-gene univariate Cox proportional hazard regression analysis was performed with the 58 GR-regulated genes using the 117-sample training set. Next, a metagene signature was optimized using the “Mystepwise” R package to select from the 58 candidates a few genes that fit into the best regression model for expression and survival of ES patients (see Methods). A signature of seven GR-regulated genes yielded statistically significant separation between low- and high-risk patients in the Kaplan-Meier analysis and the Cox proportional hazards model (FIG. 7A). The signature comprised the following genes: PDIA6, COL6A3, TMED10, SEC61G, PPA1, IGFBP7, and RPL37A. Up-regulation in response to a 60-minute long treatment of ES cells with DEX was validated with all genes, except PDIA6 and TMED10.

To confirm the findings, the predictive ability of the 7-gene signature was tested using the smaller GSE17618 cohort of 44 ES patients (validation set). According to the protocol of the training set, patients of the validation set were classified into low- and high-risk groups and Kaplan-Meier analysis was performed, while optimizing the expression threshold, to compare the differences in patient survival. Consistent with the results obtained from the training set, the 7-gene signature validated a statistically significant difference between patients with relatively short survival and the majority of patients, who survived much longer (FIG. 7B).

In summary, by applying tests of both tumor growth and metastasis and by employing three different ES models, all driven by the EWS-FLI1 oncogene, evidence was obtained that strongly associates GR with progression of ES. Mechanistically, these observations are explained by the uncovered hormone-dependent physical interactions between GR and FLI1 and the ability of the respective TF complex to enhance transcription from the glucocorticoid response element. According to the emerging model, when activated by a ligand, GR translocates to the nucleus and forms a complex with the EWS-FLI1 oncoprotein, thereby gains augmented ability to regulate transcription of anti- or pro-tumorigenic genes. Beyond the exact molecular mechanisms, the present study offers re-purposing of GR-specific antagonists and cortisol-lowering drugs for treatment of ES patients.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. Lin, K.-T. and L.-H. Wang, New dimension of glucocorticoids in cancer treatment. Steroids, 2016. 111: p. 84-88.
2. Ramamoorthy, S. and J. A. Cidlowski, Corticosteroids: Mechanisms of Action in Health and Disease. Rheumatic diseases clinics of North America, 2016. 42(1): p. 15-31, vii.
3. Rutz, H. P., Effects of corticosteroid use on treatment of solid tumours. Lancet (London, England), 2002. 360(9349): p. 1969-70.
4. Ray, A. and K. E. Prefontaine, Physical association and functional antagonism between the p65 subunit of transcription factor NF-kappa B and the glucocorticoid receptor. Proceedings of the National Academy of Sciences of the United States of America, 1994. 91(2): p. 752-6.
5. Stocklin, E., et al., Functional interactions between Stat5 and the glucocorticoid receptor. Nature, 1996. 383(6602): p. 726-8.
6. Petta, I., et al., The Interactome of the Glucocorticoid Receptor and Its Influence on the Actions of Glucocorticoids in Combatting Inflammatory and Infectious Diseases. Microbiology and molecular biology reviews: MMBR, 2016. 80(2): p. 495-522.
7. Rogatsky, I., et al., Target-specific utilization of transcriptional regulatory surfaces by the glucocorticoid receptor. Proceedings of the National Academy of Sciences of the United States of America, 2003. 100(24): p. 13845-50.
8. Murphy, S. H., et al., Tumor suppressor protein (p)53, is a regulator of NF-kappaB repression by the glucocorticoid receptor. Proceedings of the National Academy of Sciences of the United States of America, 2011. 108(41): p. 17117-22.
9. Sengupta, S., et al., Negative cross-talk between p53 and the glucocorticoid receptor and its role in neuroblastoma cells. The EMBO journal, 2000. 19(22): p. 6051-64.
10. Biddie, S. C., et al., Transcription factor AP1 potentiates chromatin accessibility and glucocorticoid receptor binding. Molecular cell, 2011. 43(1): p. 145-55.
11. Langlais, D., et al., The Stat3/GR interaction code: predictive value of direct/indirect DNA recruitment for transcription outcome. Molecular cell, 2012. 47(1): p. 38-49.
12. Lauriola, M., et al., Diurnal suppression of EGFR signalling by glucocorticoids and implications for tumour progression and treatment. Nat Commun, 2014. 5: p. 5073.
13. Yao, Z. and R. Seger, The ERK signaling cascade—views from different subcellular compartments. Biofactors, 2009. 35(5): p. 407-16.
14. Aurrekoetxea-Hernandez, K. and E. Buetti, Transforming growth factor beta enhances the glucocorticoid response of the mouse mammary tumor virus promoter through Smad and GA-binding proteins. J. Virol., 2004. 78: p. 2201-2211.
15. Michnick, S. W., et al., Universal strategies in research and drug discovery based on protein fragment complementation assays. Nature reviews Drug discovery, 2007. 6(7): p. 569-82.
16. Kovar, H., Blocking the road, stopping the engine or killing the driver? Advances in targeting EWS/FLI-1 fusion in Ewing sarcoma as novel therapy. Expert Opin Ther Targets, 2014. 18: p. 1315-28.
17. Philips, A., et al., Antagonism between Nur77 and glucocorticoid receptor for control of transcription. Molecular and cellular biology, 1997. 17(10): p. 5952-9.
18. Gilad, Y., et al., Discovering protein-protein interactions within the programmed cell death network using a protein fragment complementation screen. Cell reports, 2014. 8(3): p. 909-21.
19. Kino, T. and G. P. Chrousos, Glucocorticoid and mineralocorticoid receptors and associated diseases. Essays Biochem, 2004. 40: p. 137-55.
20. Gajulapalli, V. N. R., et al., A transcriptional repressive role for epithelial-specific ETS factor ELF3 on oestrogen receptor alpha in breast cancer cells. The Biochemical journal, 2016. 473(8): p. 1047-61.
21. Karmakar, S., Y. Jin, and A. K. Nagaich, Interaction of glucocorticoid receptor (GR) with estrogen receptor (ER) alpha and activator protein 1 (AP1) in dexamethasone-mediated interference of ERalpha activity. The Journal of biological chemistry, 2013. 288(33): p. 24020-34.
22. Garside, H., et al., Glucocorticoid ligands specify different interactions with NF-kappaB by allosteric effects on the glucocorticoid receptor DNA binding domain. The Journal of biological chemistry, 2004. 279(48): p. 50050-9.
23. Hollenhorst, P. C., L. P. McIntosh, and B. J. Graves, Genomic and biochemical insights into the specificity of ETS transcription factors. Annual review of biochemistry, 2011. 80: p. 437-71.
24. Carlson, S. M., et al., Large-scale discovery of ERK2 substrates identifies ERK-mediated transcriptional regulation by ETV3. Science signaling, 2011. 4(196): p. rs11.
25. Whirledge, S. and D. B. DeFranco, Glucocorticoid Signaling in Health and Disease: Insights From Tissue-Specific GR Knockout Mice. Endocrinology, 2018. 159(1): p. 46-64.
26. Reichardt, H. M., et al., DNA binding of the glucocorticoid receptor is not essential for survival. Cell, 1998. 93(4): p. 531-41.
27. Feng, F. Y., et al., Molecular pathways: targeting ETS gene fusions in cancer. Clinical cancer research: an official journal of the American Association for Cancer Research, 2014. 20(17): p. 4442-8.
28. Tomlins, S. A., et al., Recurrent fusion of TMPRSS2 and ETS transcription factor genes in prostate cancer. Science (New York, N Y), 2005. 310(5748): p. 644-8.
29. Tomlins, S. A., et al., Distinct classes of chromosomal rearrangements create oncogenic ETS gene fusions in prostate cancer. Nature, 2007. 448(7153): p. 595-9.
30. Soderberg, O., et al., Direct observation of individual endogenous protein complexes in situ by proximity ligation. Nature methods, 2006. 3(12): p. 995-1000.
31. Franzetti, G. A., et al., Cell-to-cell heterogeneity of EWSR1-FLI1 activity determines proliferation/migration choices in Ewing sarcoma cells. Oncogene, 2017. 36(25): p. 3505-3514.
32. Song, W., et al., Friend leukemia virus integration 1 activates the Rho GTPase pathway and is associated with metastasis in breast cancer. Oncotarget, 2015. 6(27): p. 23764-75.
33. Katschnig, A. M., et al., EWS-FLI1 perturbs MRTFB/YAP-1/TEAD target gene regulation inhibiting cytoskeletal autoregulatory feedback in Ewing sarcoma. Oncogene, 2017. 36(43): p. 5995-6005.
34. Chaturvedi, A., et al., Molecular dissection of the mechanism by which EWS/FLI expression compromises actin cytoskeletal integrity and cell adhesion in Ewing sarcoma. Molecular biology of the cell, 2014. 25(18): p. 2695-709.
35. Stoll, G., et al., Systems biology of Ewing sarcoma: a network model of EWS-FLI1 effect on proliferation and apoptosis. Nucleic acids research, 2013. 41(19): p. 8853-71.
36. Puthalakath, H., et al., The proapoptotic activity of the Bcl-2 family member Bim is regulated by interaction with the dynein motor complex. Molecular cell, 1999. 3(3): p. 287-96.
37. Luo, S., et al., Bim inhibits autophagy by recruiting Beclin 1 to microtubules. Molecular cell, 2012. 47(3): p. 359-70.
38. di Fagagna, F. D., Living on a break: cellular senescence as a DNA-damage response. Nature Reviews Cancer, 2008. 8(7): p. 512-522.
39. Linabery, A. M. and J. A. Ross, Childhood and adolescent cancer survival in the US by race and ethnicity for the diagnostic period 1975-1999. Cancer, 2008. 113(9): p. 2575-96.
40. Chaturvedi, A., et al., The EWS/FLI Oncogene Drives Changes in Cellular Morphology, Adhesion, and Migration in Ewing Sarcoma. Genes & cancer, 2012. 3(2): p. 102-16.
41. Montgomery, B., et al., Glucocorticoids and prostate cancer treatment: friend or foe? Asian journal of andrology, 2014. 16(3): p. 354-8.
42. Arora, V. K., et al., Glucocorticoid receptor confers resistance to antiandrogens by bypassing androgen receptor blockade. Cell, 2013. 155(6): p. 1309-22.
43. Herold, M. J., K. G. McPherson, and H. M. Reichardt, Glucocorticoids in T cell apoptosis and function. Cellular and molecular life sciences: CMLS, 2006. 63(1): p. 60-72.
44. Gauthier, J. M., et al., Functional interference between the Spi-1/PU.1 oncoprotein and steroid hormone or vitamin receptors. The EMBO journal, 1993. 12(13): p. 5089-96.
45. Mullick, J., et al., Physical interaction and functional synergy between glucocorticoid receptor and Ets2 proteins for transcription activation of the rat cytochrome P-450c27 promoter. J Biol Chem, 2001. 276(21): p. 18007-17.
46. Hanahan, D. and R. A. Weinberg, Hallmarks of Cancer: The Next Generation, in Cell. 2011, Elsevier. p. 646-674.
47. Ayroldi, E., et al., Mechanisms of the anti-inflammatory effects of glucocorticoids: genomic and nongenomic interference with MAPK signaling pathways. FASEB J, 2012. 26(12): p. 4805-20.
48. Ben-Chetrit, N., G. Tarcic, and Y. Yarden, ERK-ERF-EGR1, a novel switch underlying acquisition of a motile phenotype. Cell Adh Migr, 2013. 7(1): p. 33-37.
49. Kadmiel, M. and J. A. Cidlowski, Glucocorticoid receptor signaling in health and disease. Trends Pharmacol Sci, 2013. 34(9): p. 518-30.
50. Kim, S., C. T. Denny, and R. Wisdom, Cooperative DNA binding with AP-1 proteins is required for transformation by EWS-Ets fusion proteins. Molecular and cellular biology, 2006. 26(7): p. 2467-78.
51. Engblom, D., et al., Direct glucocorticoid receptor-Stat5 interaction in hepatocytes controls body size and maturation-related gene expression. Genes & development, 2007. 21(10): p. 1157-62.
52. Hunt, H. J., et al., Discovery of a novel non-steroidal GR antagonist with in vivo efficacy in the olanzapine-induced weight gain model in the rat. Bioorganic & medicinal chemistry letters, 2012. 22(24): p. 7376-80.
53. Roth, L., et al., SILAC identifies LAD1 as a filamin-binding regulator of actin dynamics in response to EGF and a marker of aggressive breast tumors. Science signaling, 2018. 11(515).
54. Miner, J. N., et al., A nonsteroidal glucocorticoid receptor antagonist. Molecular endocrinology (Baltimore, Md.), 2003. 17(1): p. 117-27.

What is claimed is:

1. A method of treating a cancer selected from the group consisting of a myeloid malignancy, a lymphoid malignancy and Ewing's sarcoma in a subject in need thereof comprising administering to the subject a therapeutically effective amount of an agent that inhibits the synthesis and/or activity of cortisol, thereby treating the cancer.

2. A method of treating a subject having a cancer characterized by expression of a fusion protein which comprises a member of the E-twenty-six (ETS) family, the method comprising:
(a) analyzing in a sample of the subject for the presence of a genomic ETS rearrangement; and
(b) administering to the subject a therapeutically effective amount of an agent that inhibits the synthesis and/or activity of cortisol upon identification of said genomic ETS rearrangement, thereby treating the cancer.

3. A method of treating a subject having a cancer characterized by expression of a fusion protein which comprises a member of the E-twenty-six (ETS) family, the method comprising:
(a) analyzing in a sample of the subject for the presence of a genomic ETS rearrangement; and
(b) administering to the subject a therapeutically effective amount of an agent that inhibits the synthesis and/or activity of cortisol upon identification of said genomic ETS rearrangement, or administering to the subject a therapeutically effective amount of an anti-cancer agent other than an agent that inhibits the synthesis and/or activity of cortisol upon identification of an absence of said genomic ETS rearrangement, thereby treating the cancer.

4. The method of claim 2, wherein said sample comprises a fluid sample.

5. The method of claim 4, wherein said fluid sample is selected from the group consisting of whole blood, plasma, serum and urine.

6. The method of claim 2, wherein said sample comprises a tissue sample.

7. The method of claim 2, wherein said analyzing for the presence of said genomic ETS rearrangement is effected at the DNA level.

8. The method of claim 2, wherein said analyzing for the presence of said genomic ETS rearrangement is effected at the RNA level.

9. The method of claim 2, wherein said analyzing for the presence of said genomic ETS rearrangement is effected at the protein level.

10. The method of claim 2, wherein said cancer is selected from the group consisting of myeloid malignancy, a lymphoid malignancy, prostate cancer and Ewing's sarcoma.

11. The method of claim 2, wherein said member of the ETS family is ERG or FL1.

12. The method of claim 2, wherein said agent that inhibits the activity of cortisol is a glucocorticoid receptor antagonist.

13. The method of claim 12, wherein said glucocorticoid receptor antagonist is a selective inhibitor of the glucocorticoid receptor.

14. The method of claim 13, wherein said selective inhibitor of the glucocorticoid receptor is C113176 or C108297.

15. The method of claim 12, wherein said glucocorticoid receptor antagonist is mifepristone.

16. The method of claim 2, wherein said agent that inhibits synthesis of cortisol is selected from the group consisting of metyrapone ketoconazole, levoketoconazole, LCI699, mitotane, aminoglutethimide and etomidate.

17. The method or agent of claim 16, wherein said agent is metyrapone.

18. The method of claim 2, wherein said agent that inhibits synthesis of cortisol is a polynucleotide agent or a proteinaceous agent that targets a component of the cortisol synthesis pathway.
